(19) United States
(12) Patent Application Publication
(10) Pub. No.: US 2022/0154204 A1
(43) Pub. Date: May 19, 2022

(54) ENHANCING DROUGHT, SALINITY AND COLD TOLERANCE IN PLANTS AND TREES

(71) Applicant: The Regents of the University of California, Oakland, CA (US)

(72) Inventors: Yohei TAKAHASHI, San Diego, CA (US); Felix HAUSER, San Diego, CA (US); Julian I. SCHROEDER, La Jolla, CA (US)

(21) Appl. No.: 17/437,586

(22) PCT Filed: Mar. 11, 2020
(86) PCT No.: PCT/US2020/022013

§ 371 Date: Sep. 9, 2021

Related U.S. Application Data
(60) Provisional application No. 62/816,492, filed on Mar. 11, 2019.

(51) Int. Cl. C12N 15/82; C12N 9/12

(52) U.S. Cl. CPC C12N 15/8273; C12N 9/12; C12Y 207/12002

(57) ABSTRACT

In alternative embodiments, provided are methods for: enhancing drought tolerance of crop plants and trees, enhancing salinity of tolerance of plants such as crop plants, enhancing early monitoring of drought, salinity and cold stress by plants such as crop plants and trees, enhancing stress resistance in plants such as crop plants and trees, by increasing the expression of or the activity of a Raf-like mitogen-activated protein (MAP) kinase kinase (MAPKK) kinase delta B3 family enzyme (or a Raf-like MAPKK kinase delta B3 family enzyme) (a M3K B3 family enzyme) in a plant or a tree cell or a plant or a tree.

### RELATED APPLICATIONS

This U.S. Utility Patent Application claims the benefit of priority under 35 U.S.C. § 119(e) of U.S. Provisional Application Ser. No. 62/816,492, Mar. 11, 2019. The aforementioned application is expressly incorporated herein by reference in its entirety and for all purposes.

### STATEMENT AS TO FEDERALLY SPONSORED RESEARCH

This invention was made with government support under ES010337 and GM060396 awarded by the National Institutes of Health and under MCB-1900567 awarded by the National Science Foundation. The government has certain rights in the invention.

### TECHNICAL FIELD

This invention generally relates to agriculture and molecular biology. In alternative embodiments, provided are methods for: enhancing drought tolerance of crop plants and trees, enhancing salinity of tolerance of plants such as crop plants, enhancing early monitoring of drought, salinity and cold stress by plants such as crop plants and trees, enhancing stress resistance in plants such as crop plants and trees, by increasing the expression of or the activity of a Raf-like mitogen-activated protein (MAP) kinase kinase (MAPKK) kinase δ B3 family enzyme (or a Raf-like MAPKK kinase δ B3 family enzyme) (an M3K δ B3 family enzyme) in a plant or a tree cell or a plant or a tree.

### BACKGROUND

The response of plants to reduced water availability is controlled by a complex osmotic stress and abscisic acid (ABA)-dependent signal transduction network. The core ABA signaling components are snf1-related protein kinase2s (SnRK2s) which are activated by ABA-dependent inhibition of type 2C protein phosphatases and by an unknown ABA-independent osmotic stress signaling pathway.

Limited water availability is one of the key factors that negatively impacts crop yields. The plant hormone abscisic acid (ABA) and the signal transduction network it activates, enhance plant drought tolerance through stomatal closure, and inhibition of seed germination and growth (Finkelstein, 2013). As plants are constantly exposed to changing water conditions, reversibility and robustness of the ABA signal transduction cascade is important for plants to balance growth and drought stress resistance. Core ABA signaling components have been established (Ma et al., 2009; Park et al., 2009): the ABA receptors PYRABACTIN RESISTANCE (PYR/PYL) or REGULATORY COMPONENT OF ABA RECEPTOR (RCAR) inhibit type 2C protein phosphatases (PP2Cs) (Ma et al., 2009; Park et al., 2009), resulting in the activation of the SnRK2 protein kinases SnRK2.2, 2.3 and OST1/SnRK2.6 (Umezawa et al., 2009; Vlad et al., 2009). The SnRK2 kinases phosphorylate and thus regulate the activity of downstream components such as ion channels and transcription factors (Fujii et al., 2009; Geiger et al., 2009; Lee et al., 2009; Takahashi et al., 2013), which leads to stomatal closure and changes in gene expression. Activation of SnRK2 protein kinases requires phosphorylation of the SnRK2 kinases themselves, and in vitro experiments using purified recombinant OST1/SnRK2.6 suggest that phosphorylation of the activation-loop is an important step (Belin et al., 2006). However, it has remained unclear whether direct auto-phosphorylation or trans-phosphorylation by unknown protein kinases re-activates these SnRK2 protein kinase in response to stress.

Previous studies showed that ABA-dependent phosphorylation of substrate proteins of SnRK2 could be reconstituted in vitro using only recombinant PYR/RCAR ABA receptors, PP2Cs and SnRK2 proteins. (Fujii et al., 2009; Brandt et al., 2012; Takahashi et al., 2017). Recombinant SnRK2 proteins used in these studies, unlike SnRK2s in plant cells, had high intrinsic kinase activities even before ABA treatment. Therefore it is not clear whether autophosphorylation accounts for the ABA-dependent SnRK2 reactivation after PP2C-dependent inhibition.

The Arabidopsis genome encodes ten SnRK2 kinases, and at least nine of them are activated in response to osmotic stress (Boudsocq et al., 2004). Interestingly, osmotic stress-induced activation of SnRK2 protein kinases can occur independently of ABA signaling (Yoshida et al., 2006). The osmotic stress sensing mechanism and upstream signal transduction mechanisms leading to SnRK2 activation remain largely unknown in plants.

### SUMMARY

In alternative embodiments, provided are methods for
enhancing or creating drought tolerance of crop plants and trees,
enhancing or creating salinity of tolerance of a plant, wherein optionally the plant is a crop plant,
early monitoring of drought, salinity and cold stress by a plant or a tree, wherein optionally the plant is a crop plant, or
enhancing or creating stress resistance in a plant or a tree, wherein optionally the plant is a crop plant,

the method comprising increasing the expression and/or activity of a Raf-like mitogen-activated protein (MAP) kinase kinase (MAPKK) kinase δ B3 family enzyme (an M3K δ B3 family enzyme) in a plant or a tree, or a plant cell or a tree cell by: inserting in the a plant or a tree, or a plant cell or a tree cell, a heterologous M3K δ B3 family enzyme-expressing nucleic acid, wherein the nucleic acid is operatively linked to a transcriptional regulatory element that is capable of expressing the M3K δ B3 family enzyme in the plant or tree or plant cell or tree cell, resulting in increasing the amount of M3K δ B3 family enzyme expression or M3K δ B3 family enzyme activity in the plant or tree or plant cell or tree cell.

The method of claim 1, wherein the transcriptional regulatory element comprises a promoter, and optionally the promoter comprises an inducible promoter, a constitutive promoter, a guard cell specific promoter, a drought-inducible promoter, a stress-inducible promoter or a guard cell active promoter, and optionally the promoter comprises: a pRAB18 drought and ABA-induced promoter; a pGC1 guard cell promoter; a constitutive CAMV 35 promoter; a constitutive pUbi10 promoter.

In alternative embodiments, of methods as provided herein:
the method comprises increasing the total or average amount of M3K δ B3 family enzyme expression or M3K δ B3 family enzyme activity in the plant or tree or plant cell or tree cell by between about 5% and 500%, or by between about 10% and 200%;
the plant or tree is, or the plant or tree cell is derived from: (i) a dicotyledonous or monocotyledonous plant; (ii) wheat, oat, rye, barley, rice, sorghum, maize (corn), tobacco, a legume, a lupins, potato, sugar beet, pea, bean, soybean (soy), a cruciferous plant, a cauliflower, rape (or rapa or canola), cane (sugarcane), flax, cotton, palm, sugar beet, peanut, a tree, a poplar, a lupin, a silk cotton tree, desert willow, creosote bush, winterfat, balsa, ramie, kenaf, hemp, roselle, jute, or sisal abaca; or, (c) a species from the genera Arabidopsis, Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Man[iota]hot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna or Zea;
the M3K δ B3 family enzyme or M3K δ B3 family enzyme-expressing nucleic acid is or comprises:
   (a) a rice (e.g., of the genus Oryza, or Oryza sativa) M3K δ B3 enzyme or enzyme-encoding nucleic acid having a sequence as set forth in GenBank accession no. AY167575.1; XP_015625387.1; EEC72758.1; EEE56573.1; BAH01506.1 or XP_015636565.1;
   (b) a soybean (e.g., of the genus Glycine, or G. max) M3K δ B3 enzyme or enzyme-encoding nucleic acid having a sequence as set forth in GenBank accession no. FJ528664.1; XP_003545374; KRH34026.1; ACQ57002.1; XP 006578285.1 or XP 006596381.1; or
   (c) a maize (e.g., of the genus Zea, or Zea mays) CM007647.1; XP_008679833.1; AQK59735.1; AQK59729.1; KQJ94060.1 or XP_008668902.1; and/or
the heterologous M3K δ B3 family enzyme-expressing nucleic acid is contained in an expression vector, which is optionally an episome, or is the heterologous M3K δ B3 family enzyme-expressing nucleic acid is stably integrated into the plant, tree, plant cell or tree cell genome.

In alternative embodiments, provided are transgenic guard cells, plants, plant cells, plant tissues, plant seeds or fruits, or plant parts or plant organs that expresses a heterologous M3K δ B3 family enzyme, comprising: a heterologous M3K δ B3 family enzyme-expressing nucleic acid operatively linked to transcriptional regulatory element, and optionally the transcriptional regulatory element comprises a promoter, and optionally the promoter comprises an inducible promoter, a constitutive promoter, a guard cell specific promoter, a drought-inducible promoter, a stress-inducible promoter or a guard cell active promoter, and optionally the promoter comprises: a pRAB18 drought and ABA-induced promoter; a pGC1 guard cell promoter; a constitutive CAMV 35 promoter; a constitutive pUbi10 promoter.

In alternative embodiments, provided are uses of transgenic guard cells, plants, plant cells, plant tissues, plant seeds or fruits, or plant parts or plant organs as provided herein for: enhancing or creating drought tolerance of crop plants and trees, enhancing or creating salinity of tolerance of a plant, wherein optionally the plant is a crop plant, early monitoring of drought, salinity and cold stress by a plant or a tree, wherein optionally the plant is a crop plant, or, enhancing or creating stress resistance in a plant or a tree, wherein optionally the plant is a crop plant.

In alternative embodiments, provided are transgenic guard cells, plants, plant cells, plant tissues, plant seeds or fruits, or plant parts or plant organs as provided herein for use in: enhancing or creating drought tolerance of crop plants and trees, enhancing or creating salinity of tolerance of a plant, wherein optionally the plant is a crop plant, early monitoring of drought, salinity and cold stress by a plant or a tree, wherein optionally the plant is a crop plant, or, enhancing or creating stress resistance in a plant or a tree, wherein optionally the plant is a crop plant.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1A-G illustrates the identification of M3Ks that reactivate OST1/SnRK2 kinases by phosphorylation: FIG. 1A illustrates images of seeds of amiR-HsMYO wild-type (control line) or amiR-ax1117 mutant were sowed on ½ MS medium containing 2 μM ABA, or 0.02% EtOH as control, for germination assays, representative images showing seed germination after 6 days; FIG. 1B graphically illustrates the percentage of seedlings showing green cotyledons was analyzed, each experiment included 64 seeds for each genotype, letters at the top of columns are grouped based on two-way ANOVA and Tukey's test; FIG. 1C illustrates identification of the amiRNA sequence in amiR-ax1117 plants, the black box labels the sequence of amiR-ax1117, which is the underlined

(SEQ ID NO: 18)
CTTTGATCCAAAATCGAAACCTTCACTCTC;

FIG. 1D illustrates data from a study where wild type (WT) and m3k amiRNA seedlings were incubated with 10 μM ABA for 15 min, and in-gel kinase assays were performed using histone type III-S as a substrate; FIG. 1E illustrates SnRK2 band intensities as shown in FIG. 1d, were measured using ImageJ; FIG. 1F illustrates a study where recombinant GST-OST1/SnRK2.6 protein was dephosphorylated by alkaline phosphatase in vitro and used for in vitro phosphorylation assays after incubation with CPK6, CPK23 or MPK12 protein kinases; FIG. 1G illustrates a study where dephosphorylated recombinant His-OST1/SnRK2.6 protein was incubated with kinase domains of seven M3Ks and used for in-gel kinase assays; all as described in further detail in Example 1, below.

FIG. 2A-E illustrates data showing that M3K-dependent OST1/SnRK2.6 Ser-171 phosphorylation is essential for ABA activation of OST1/SnRK2.6 activation: FIG. 1A illustrates that the inactive M3K66 kinase domain mutant (K775W) did not re-activate His-OST1/SnRK2.6 in vitro; FIG. 2B illustrates a study where inactive GST-OST1/SnRK2.6-D140A kinase protein was incubated with M3Kδ6 kinase domain, and in vitro phosphorylation assays were performed with ³²P-ATP; FIG. 2C illustrates a study where recombinant inactive OST1(D140A) and M3Kδ1 kinase domains were incubated with ATP, and a mass spectrum of phosphorylated OST1 peptide (SSVLHpSQPK) is shown; FIG. 2D illustrates a study where phosphorylation at Ser171 was not detectable after in vitro auto-phosphorylation of OST1/SnRK2.6, but was consistently phosphorylated in the presence of M3Kδ1; FIG. 1E illustrates a study where OST1(S171A)-GFP was transiently expressed in Arabidopsis mesophyll cell protoplasts; all as described in further detail in Example 1, below.

FIG. 3A-F illustrates data showing that OST1/SnRK2.6 Ser171 is essential for ABA-induced stomatal closure and S-type anion channel activation in planta: FIG. 3A illustrates studies where stomatal conductances were analyzed in intact detached leaves of stable transgenic Arabidopsis; FIG. 3B illustrates studies of leaf temperatures of Col, ost1-3, OST1-comp2 and S171A-comp2, as measured by thermal imaging; FIG. 3C illustrates studies where leaf temperatures were measured by using Fiji software; FIG. 3D illustrates studies where ABA-activated S-type anion channel currents were investigated by patch-clamp analyses using guard cell protoplasts from the transgenic Arabidopsis lines; FIG. 3E illustrates studies of the average current voltage relationship of S-type anion channel as shown; FIG. 3F illustrates kinase activities of OST1(S171A) in mesophyll cells from stably-transformed homozygous transgenic plants were investigated by in-gel kinase assays; all as described in further detail in Example 1, below.

FIG. 4A-F illustrates studies showing that MAPKK-kinases are essential for ABA signalling module: FIG. 4A-B illustrates studies of in vitro reconstitution of ABA-induced OST1/SnRK2 activation without M3Kδ6 (FIG. 4A) or with M3Kδ6 (FIG. 4B); FIG. 4C illustrates studies where recombinant His-PYR1/RCAR11, His-HAB1, His-OST1/SnRK2.6, His-AKS1 and GST-M3Kδ6 kinase domain were mixed as indicated above the gel; FIG. 4D-F illustrates studies of reconstitution of ABA-activation of SLAC1 channels in Xenopus oocytes, in the presence or absence of M3Ks, FIG. 4D illustrates representative whole cell chloride current recordings of oocytes co-expressing the indicated proteins, without (control) or with injection of 50 μM ABA (+ABA), FIG. 4E illustrates mean current-voltage curves of oocytes co-expressing the indicated proteins, with or without injection of ABA, FIG. 4F illustrates average SLAC1-mediated currents at −100 mV, co-expressing the indicated proteins, in the presence or absence of 50 μM ABA; all as described in further detail in Example 1, below.

FIG. 5A-I illustrates studies showing that MAPKK-kinases are required for plant ABA response: FIG. 5A illustrates genome structures and T-DNA insertion sites of M3K genes; FIG. 5B illustrates genomic regions of CRISPR/Cas9-mediated M3Kδ1 and M3Kδ7 gene deletions; FIG. 5C illustrates studies of RT-PCR assays showing transcripts of kinase domains of M3Ks in the m3kδ1crispr m3kδ6-2 m3kδ7crispr triple mutant; FIG. 5D illustrates studies of m3kδ1crispr m3kδ6-2 m3kδ7crispr triple mutant seedlings were grown on ½ MS plates supplemented with 2 μM ABA or ethanol (control) for 16 days; FIG. 5E illustrates studies where seedlings showing green cotyledons as in (FIG. 5D) were counted; FIG. 5F illustrates studies of RT-PCR which show M3Kδ1, δ6 and δ7 expression in the indicated m3k T-DNA insertion mutants; FIG. 5G illustrates studies of m3kδ1 m3kδ6-1 m3kδ7 T-DNA triple mutant plants were grown on ½MS plates supplemented with 0.8 μM ABA for 9 days; FIG. 5H illustrates data where seedlings showing green cotyledons as in (FIG. 5G) were counted; FIG. 5I illustrates studies where three amiRNA lines targeting M3Kδ1, 66 and 67 were grown on ½MS plates supplemented with EtOH (control) or 2 μM ABA for 9 days, and as a control line, the amiRNA-HsMYO line²¹ was used;

FIG. 5J illustrates studies where seedlings showing green cotyledons as in (FIG. 5I) were counted; all as described in further detail in Example 1, below.

FIG. 6A-F illustrates studies showing that MAPKK-kinases mediate ABA- and osmotic stress-induced SnRK2 activation in planta: FIG. 6A illustrates studies where m3kδ1crispr m3kδ6-2 m3kδ7crispr triple mutant seedlings were incubated in 10 μM ABA or 0.3 M mannitol (Osmo) for 15 min, SnRK2 activities were tested by in-gel kinase assays, arrowhead shows SnRK2 activity²³; FIG. 6B graphically illustrates normalized band intensities as shown in (FIG. 6A) measured by using ImageJ, SnRK2 activities were analyzed by in-gel kinase assays; FIG. 6C illustrates studies where m3kδ1 m3kδ6-1 m3kδ7 T-DNA triple mutant seedlings were incubated in 10 μM ABA or 0.3 M mannitol (Osmo) for 15 min, SnRK2 activities were analyzed by in-gel kinase assays; FIG. 6D illustrates studies where normalized band intensities as shown in (FIG. 6C) were measured by using ImageJ; FIG. 6E illustrates studies where recombinant GST-tagged Arabidopsis SnRK2 protein kinases were incubated with M3Kδ1 kinase domain, SnRK2 kinase activities were analyzed by in-gel kinase assays; FIG. 6F illustrates studies where M3Kδ6-FLAG was transiently expressed in Arabidopsis mesophyll cell protoplasts; all as described in further detail in Example 1, below.

FIG. 7 illustrates a schematic of a phylogenetic tree of Arabidopsis Raf-like MAPKK kinases, as described in further detail in Example 1, below.

FIG. 8 illustrates a biological replicate example of SnRK2 activities in m3k amiRNA line, where a replicate of an in-gel kinase assay using m3k amiRNA line and WT (Col-0 accession) is shown, as described in further detail in Example 1, below.

FIG. 9 illustrates that Full-length M3Kδ1 activates OST1/SnRK2.6 in vitro, where recombinant GST-OST1 and full-length His-M3Kδ1 (M3Kδ1_Full) or GST-M3Kδ1_KD (kinase domain) were incubated in the presence of ATP for 30 min, and OST1/SnRK2.6 activity was measured by in-gel kinase assays, as described in further detail in Example 1, below.

FIG. 10 illustrates studies where M3Kδ1, 66 and 67 directly phosphorylate OST1/SnRK2.6, and recombinant kinase inactive GST-OST1/SnRK2.6 (D140A) protein was incubated with M3Kδ1, M3Kδ6 or M3Kδ7 kinase domains, and in vitro phosphorylation assays were performed with ³²P-ATP, as described in further detail in Example 1, below.

FIG. 11A-C illustrates that Ser-171 is important for ABA-activation of OST1/SnRK2.6 but not for in vitro protein kinase enzyme activity: FIG. 11A illustrates studies where recombinant GST-OST1/SnRK2.6 proteins carrying S171A, S175A or T176A mutation were used for in vitro autophosphorylation; FIG. 11B illustrates studies where OST1/SnRK2.6-GFP variants (WT, S171A, S175A or T176A) were transiently expressed in Arabidopsis mesophyll cell protoplasts, protoplasts were incubated in 10 μM ABA for 15 min, and OST1/SnRK2.6 protein kinase activity was detected by in-gel kinase assays (top panel) and OST1/SnRK2.6-GFP proteins were detected by immuno-blot using GFP antibody (bottom panel); FIG. 11C illustrates studies where OST1-GFP (S171E) kinase activity was tested as shown in (FIG. 11B), as described in further detail in Example 1, below.

FIG. 12A-B illustrates studies showing that ABA induces phosphorylation at Ser-171 in OST1/SnRK2.6: FIG. 12A illustrates studies where the sequence of identified phosphorylated peptide (SSVLHSQPKSTVGTPAYIAPEVLLK (SEQ ID NO:19)) was identified by mass spectrometry; FIG. 11B illustrates where annotated mass spectrum of the phosphorylated peptide (SEQ ID NO:19) in the presence of ABA, as described in further detail in Example 1, below.

FIG. 13A-D illustrates studies showing that stomatal conductances and leaf temperatures of independent transgenic ost1-3 Arabidopsis lines expressing OST1(WT or S171A)-HF: FIG. 13A illustrates studies where stomatal conductances were analyzed in detached leaves of stable transgenic Arabidopsis; FIG. 13B illustrates studies where relative stomatal conductances in (FIG. 13A) were normalized to the average of the 10 minutes before addition of ABA; FIG. 13C illustrates studies where oeaf temperatures of homozygous transgenic Arabidopsis lines (OST1_comp1 and S171A_comp1) were measured by thermal imaging; FIG. 13D illustrates studies where leaf temperatures were measured by using Fiji software, all as described in further detail in Example 1, below.

FIG. 14A-G illustrates studies showing that M3Kδs activate SLAC1 channels together with OST1/SnRK2.6 in Xenopus oocytes: FIG. 14A illustrates representative whole cell chloride current recordings of oocytes injected with the indicated cRNAs: FIG. 14B-D illustrates mean current-voltage curves of oocytes co-expressing OST1 and SLAC1, in the presence or absence of the indicated M3K proteins; FIG. 14E illustrates average SLAC1 mediated currents at −100 mV, co-expressing OST1, in the presence or absence of the indicated M3K proteins; FIG. 14F illustrates mean current-voltage curves of Xenopus oocytes injected with the indicated cRNAs; FIG. 14G illustrates average SLAC1-mediated currents from FIG. 14F, at −120 mV, co-expressing OST1 isoforms, in the presence of M3Kδ1, all as described in further detail in Example 1, below.

FIG. 15A-E illustrates studies showing that M3K-dependent S-type anion channel activation is dependent on the M3K kinase activities and the Ser171 residue in the OST1/SnRK2.6 activation loop: FIG. 15A-D illustrate mean current-voltage curves of Xenopus oocytes injected with the indicated cRNAs; FIG. 14E graphically illustrates the results of the data from FIG. 14A-D, all as described in further detail in Example 1, below.

FIG. 16A-H illustrates studies showing that ABA-induced stomatal closing and activation of guard cell S-type anion channels are impaired m3k amiRNA line: FIG. 16A illustrates studies where leaves from m3k amiRNAi and the control amiRNA-HsMYO line (expressing an amiRNA targeting human myosin 2), which has no target gene in Arabidopsis plants were analyzed in time-resolved stomatal conductance analyses in which 1 μM ABA was added to the transpiration stream via the petiole; FIG. 16B illustrates normalized relative stomatal conductance to the first data point shown in (FIG. 16A); FIG. 16C-H illustrates studies where ABA-activated S-type anion channel currents were investigated by patch-clamp analyses using guard cell protoplasts from the wildtype parent Col-0 (FIG. 15C-D), the HsMYO amiRNA control line (FIG. 15E-F), and the m3k amiRNA line (FIG. 15G-H), and representative current traces (FIG. 15C, FIG. 15E, FIG. 15G) and average current voltage relationships (FIG. 15D, FIG. 15F, FIG. 15H) of S-type anion channel currents are shown, all as described in further detail in Example 1, below.

FIG. 17A-C illustrates studies showing that m3k double mutants show weak ABA-insensitive phenotypes: FIG. 17A illustrates studies where m3k double (m3kδ6-2 87) and triple (m3kδ1/δ6-1/δ7) mutants were grown on ½ MS plates supplemented with 2 μM ABA or EtOH for 9 days, and seedlings showing green cotyledons were counted; FIG. 17B illustrates studies where m3k double (m3kδ1/δ7) and triple (m3kδ1crispr δ6-2/δ7crispr) mutants were grown on ½ MS plates supplemented with 2 μM ABA or EtOH for 16 days, and seedlings showing green cotyledons were counted; FIG. 17C illustrates studies where wild type and m3kδ1/δ6-1/δ7 triple mutant seedlings were grown on ½ MS plates for three days and transferred to ½ MS plates with or without 20 μM ABA followed by an additional seven-day incubation, and primary root length was measured using ImageJ software, all as described in further detail in Example 1, below.

FIG. 18 illustrates a biological replicate example of SnRK2 activities in m3kδ1/δ6-1/δ7 triple mutant line, a replicate of in-gel kinase assay using m3kδ1/δ6-1/δ7 triple mutant is shown, as described in further detail in Example 1, below.

FIG. 19A-B illustrates studies showing that m3k mutants show reduced sensitivity to osmotic stress in seed germination: FIG. 19A illustrates studies where m3kδ1/δ87 double mutant and m3kδ1crispr δ6-2/δ7crispr triple mutant seedlings were grown on ½ MS plates supplemented with 0.4 M mannitol for 3 days and green cotyledons were counted; FIG. 19B illustrates studies where three amiRNA lines targeting M3Kδ1, δ6 and δ7 were grown on ½ MS plate supplemented with 0.4 M mannitol for 3 days, all as described in further detail in Example 1, below.

FIG. 20A-B illustrates studies showing that M3Ks activate SnRK2.3 in vitro: FIG. 20A illustrates studies where GST-SnRK2.3 protein was incubated with the kinase domains of M3Kδ1, M3Kδ6 or M3Kδ7 and in-gel kinase assays were conducted; FIG. 20B illustrates studies where SnRK2.2-GFP (WT or S180A) and SnRK2.3-GFP (WT or S172A) were expressed in Arabidopsis mesophyll cell protoplasts and purified by immunoprecipitation with GFP antibodies, all as described in further detail in Example 1, below.

FIG. 21A-D: illustrates studies showing that M3Ks interact with SnRK2 kinases in BiFC experiments in plant cells: FIG. 21A illustrates co-immunoprecipitation experiments using transiently expressed M3Kδ6 and OST1/SnRK2.6 in Arabidopsis mesophyll cell protoplasts, and OST1/SnRK2.6-GFP or GFP control co-expressed with M3Kδ6-FLAG were immunoprecipitated with GFP antibodies, and precipitated proteins were analyzed by immunoblots using GFP or FLAG antibody;

FIG. 21B-C illustrate BiFC analyses of nYFP-M3Kδ6 (FIG. 21B) or nYFP-M3Kδ7 (FIG. 21C) with OST1/SnRK2.6-cYFP, SnRK2.2-cYFP, 2.4-cYFP and 2.10-cYFP infiltrated in 6-week-old Nicotiana benthamiana leaves; FIG. 21D-E illustrate BiFC quantifications measured from maximal projections of z-stacks and normalized over an infiltration control expressing p19 only, all as described in further detail in Example 1, below.

FIG. 22: A-B illustrates studies showing that m3k quadruple mutant shows an ABA-insensitive phenotype in cotyledon emergence; FIG. 22A illustrates studies where m3k triple (m3kδ1/δ6-1/δ7) and m3k quadruple (m3kδ1/δ5/δ6-1/δ7) mutant plants were grown on ½ MS plates supplemented with 0.8 μM ABA for 6 days, and green emerging cotyledons were counted; FIG. 22B illustrates gene expression levels of B3 subgroup M3K genes and three SnRK2 genes in guard cells and mesophyll cells Like reference symbols in the various drawings indicate like elements, all as described in further detail in Example 1, below.

FIG. 23 illustrates primer sequences used for cloning in studies described in Example 1, below.

### DETAILED DESCRIPTION

In alternative embodiments, provided are methods for: enhancing drought tolerance of crop plants and trees, enhancing salinity of tolerance of plants such as crop plants, enhancing early monitoring of drought, salinity and cold stress by plants such as crop plants and trees, enhancing stress resistance in plants such as crop plants and trees, by increasing the expression of or the activity of a Raf-like mitogen-activated protein (MAP) kinase kinase (MAPKK) kinase δ B3 family enzyme (or a Raf-like MAPKK kinase δ B3 family enzyme) (an M3K δ B3 family enzyme) in a plant or a tree cell or a plant or a tree.

In alternative embodiments, provided are methods for enhancing plant and tree drought and salinity stress resistance and protect yields of plants such as crop plants and trees exposed to drought stress. In alternative embodiments, provided are methods for enhancing drought and salinity tolerance of plants such as crop plants and trees. In alternative embodiments, provided are methods for enhancing early monitoring of drought and salinity-linked osmotic stress in plants such as crop plants and trees, which can boost early mounting of stress resistance in the plants and trees.

Through a combination of a redundancy-circumventing genetic screen and biochemical analyses, we have identified key functionally-redundant Raf-like MAPKK kinases (M3Ks) that activate OST1/SnRK2 kinases. Reactivation of dephosphorylated SnRK2 requires these M3Ks, and ABA-induced OST1/SnRK2.6 activation and S-type anion channel activation requires the presence of M3Ks. M3K knock-out plants show not only reduced sensitivity to ABA but also strongly impaired osmotic stress-induced SnRK2 activation. Our results demonstrate that these Raf-like M3Ks are required for ABA- and osmotic stress-activation of SnRK2 kinases, ensuring robust ABA and osmotic stress signal transduction and indicate that increased resistance to these stresses can be engineered through targeted over-expression and enhancement of these protein activities.

This newly recognized mechanism can be used to boost both drought and salinity osmotic stress sensing as well abscisic acid drought and temperature resistance responses in plants.

The described advances can be used via over-expression of the identified mechanisms using promoters and/or genome editing using several genome editing platforms, including non-restricted technologies, to: enhance drought tolerance of crop plants and trees; enhance salinity of tolerance of crop plants; enhance early monitoring of drought, salinity and cold stress by crop plants and trees; provide for early mounting of stress resistance in crop plants and trees.

Genome editing can be accomplished using transcription activator-like effector nuclease (TALEN) gene editing, see e.g., Zhang et al Plant Physiology (2013) vol 161(1):pg 20-27; Haun et al (2014) Plant Biotechnology Journal, Vol 12(7): 934-40; or unrestricted TALEN™ (ThermoFisher) technology.

### Plant (Expressible) Promoters

In alternative embodiments, promoters that can be used to drive the over-expression of an M3K δ B3 family enzyme in a plant or a tree cell or a plant or a tree for practicing exemplary methods as provided herein, including enhancing drought and salinity tolerance, comprise: a pRAB18 drought and ABA-induced promoter; a pGC1 guard cell promoter; a constitutive CAMV 35 promoter; a constitutive pUbi10 promoter.

In alternative embodiments, M3K nucleic acids and M3K-protein coding sequences or genes used to practice methods as provided herein are operably linked to a plant expressible promoter, an inducible promoter, a constitutive promoter, a guard cell specific promoter, a drought-inducible promoter, a stress-inducible promoter or a guard cell active promoter. Promoters used to practice methods as provided herein include a strong promoter, particularly in plant guard cells, and in some embodiments is guard cell specific, e.g., the promoters described in WO2008/134571.

In alternative embodiments, M3K nucleic acids and M3K-protein coding sequences or genes also can be operatively linked to any constitutive and/or plant specific, or plant cell specific promoter, e.g., a cauliflower mosaic virus (CaMV) 35S promoter, a mannopine synthase (MAS) promoter, a 1′ or 2′ promoter derived from T-DNA of Agrobacterium tumefaciens, a figwort mosaic virus 34S promoter, an actin promoter, a rice actin promoter, a ubiquitin promoter, e.g., a maize ubiquitin-1 promoter, and the like.

Examples of constitutive plant promoters which can be useful for expressing the M3K-encoding sequences in accordance with methods as provided herein include: the cauliflower mosaic virus (CaMV) 35S promoter, which confers constitutive, high-level expression in most plant tissues (see, e.g., Odell et al. (1985) Nature 313: 810-812); the nopaline synthase promoter (An et al. (1988) Plant Physiol. 88: 547-552); and the octopine synthase promoter (Fromm et al. (1989) Plant Cell 1: 977-984).

A variety of plant gene promoters that regulate gene expression in response to environmental, hormonal, chemical, developmental signals, and in a tissue-active manner can be used for expression of a sequence in plants. Choice of a promoter is based largely on the phenotype of interest and is determined by such factors as tissue (e.g., seed, fruit, root, pollen, vascular tissue, flower, carpel, etc.), inducibility (e.g., in response to wounding, heat, cold, drought, light, pathogens, etc.), timing, developmental stage, and the like.

Numerous known promoters have been characterized and can be employed to promote expression of a polynucleotide used to practice methods as provided herein, e.g., in a transgenic plant or cell of interest. For example, tissue specific promoters include: seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2Al 1 promoter (e.g., see U.S. Pat. No. 4,943,674) and the tomato polygalacturonase promoter (e.g., see Bird et al. (1988) Plant MoI. Biol. 11: 651-662), root-specific promoters, such as those disclosed in U.S. Pat. Nos. 5,618,988, 5,837,848 and 5,905,186, pollen-active promoters such as PTA29, PTA26 and PTAl 3 (e.g., see U.S. Pat. No. 5,792,929), promoters active in vascular tissue (e.g., see Ringli and Keller (1998) Plant MoI. Biol. 37: 977-988), flower-specific (e.g., see Kaiser et al. (1995) Plant MoI. Biol. 28: 231-243), pollen (e.g., see Baerson et al. (1994) Plant MoI. Biol. 26: 1947-1959), carpels (e.g., see OhI et al. (1990) Plant Cell 2: pollen and ovules (e.g., see Baerson et al. (1993) Plant MoI. Biol. 22: 255-267), auxin-inducible promoters (such as that described in van der Kop et al. (1999) Plant MoI. Biol. 39: 979-990 or Baumann et al., (1999) Plant Cell 11: 323-334), cytokinin-inducible promoter (e.g., see Guevara-Garcia (1998) Plant MoI. Biol. 38: 743-753), promoters responsive to gibberellin (e.g., see Shi et al. (1998) Plant MoI. Biol. 38: 1053-1060, Willmott et al. (1998) Plant Molec. Biol. 38: 817-825) and the like.

Additional promoters that can be used to practice methods as provided herein are those that elicit expression in response to heat (e.g., see Ainley et al. (1993) Plant MoI. Biol. 22: 13-23), light (e.g., the pea rbcS-3A promoter, Kuhlemeier et al. (1989) Plant Cell 1: 471-478, and the maize rbcS promoter, Schaffher and Sheen (1991) Plant Cell 3: 997-1012); wounding (e.g., wunl, Siebertz et al. (1989) Plant Cell 1: 961-968); pathogens (such as the PR-I promoter described in Buchel et al. (1999) Plant MoI. Biol. 40: 387-396, and the PDF 1.2 promoter described in Manners et al. (1998) Plant MoI. Biol. 38: 1071-1080), and chemicals such as methyl jasmonate or salicylic acid (e.g., see Gatz (1997) Annu. Rev. Plant Physiol. Plant MoI. Biol. 48: 89-108). In addition, the timing of the expression can be controlled by using promoters such as those acting at senescence (e.g., see Gan and Amasino (1995) Science 270: 1986-1988); or late seed development (e.g., see Odell et al. (1994) Plant Physiol. 106: 447-458).

In alternative embodiments, tissue-specific and/or developmental stage-specific promoters are used, e.g., promoter that can promote transcription only within a certain time frame of developmental stage within that tissue. See, e.g., Blazquez (1998) Plant Cell 10:791-800, characterizing the Arabidopsis LEAFY gene promoter. See also Cardon (1997) Plant J 12:367-77, describing the transcription factor SPL3, which recognizes a conserved sequence motif in the promoter region of the A. thaliana floral meristem identity gene API; and Mandel (1995) Plant Molecular Biology, Vol. 29, pp 995-1004, describing the meristem promoter eIF4. Tissue specific promoters which are active throughout the life cycle of a particular tissue can be used. In one aspect, M3K-encoding nucleic acids used to practice methods as provided herein are operably linked to a promoter active primarily only in cotton fiber cells, hi one aspect, M3K-encoding nucleic acids used to practice methods as provided herein are operably linked to a promoter active primarily during the stages of cotton fiber cell elongation, e.g., as described by Rinehart (1996) supra. The M3K-encoding nucleic acids used to practice methods as provided herein can be operably linked to the Fbl2A gene promoter to be preferentially expressed in cotton fiber cells (Ibid). See also, John (1997) Proc. Natl. Acad. Sci. USA 89:5769-5773; John, et al., U.S. Pat. Nos. 5,608,148 and 5,602,321, describing cotton fiber-specific promoters and methods for the construction of transgenic cotton plants. Root-specific promoters may also be used to express M3K-encoding nucleic acids used to practice methods as provided herein. Examples of root-specific promoters include the promoter from the alcohol dehydrogenase gene (DeLisle (1990) Int. Rev. Cytol. 123:39-60). Other promoters that can be used to express M3K-encoding nucleic acids used in methods as provided herein include, e.g., ovule-specific, embryo-specific, endosperm-specific, integument-specific, seed coat-specific promoters, or some combination thereof, a leaf-specific promoter (see, e.g., Busk (1997) Plant J. 11:1285 1295, describing a leaf-specific promoter in maize); the ORF 13 promoter from Agrobacterium rhizogenes (which exhibits high activity in roots, see, e.g., Hansen (1997) supra); a maize pollen specific promoter (see, e.g., Guerrero (1990) MoI. Gen. Genet. 224:161 168); a tomato promoter active during fruit ripening, senescence and abscission of leaves and, to a lesser extent, of flowers can be used (see, e.g., Blume (1997) Plant J. 12:731 746); a pistil-specific promoter from the potato SK2 gene (see, e.g., Ficker (1997) Plant MoI. Biol. 35:425 431); the Blec4 gene from pea, which is active in epidermal tissue of vegetative and floral shoot apices of transgenic alfalfa making it a useful tool to target the expression of foreign genes to the epidermal layer of actively growing shoots or fibers; the ovule-specific BEL1 gene (see, e.g., Reiser (1995) Cell 83:735-742, GenBank No. U39944); and/or, the promoter in Klee, U.S. Pat. No. 5,589,583, describing a plant promoter region is capable of conferring high levels of transcription in meristematic tissue and/or rapidly dividing cells.

In alternative embodiments, plant promoters used in methods as provided herein can be inducible upon exposure to plant hormones, such as auxins; these promoters can be used to express M3K nucleic acids used in methods as provided herein. For example, exemplary methods can use the auxin-response elements El promoter fragment (AuxREs) in the soybean {Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) MoI. Plant Microbe Interact. 10:933-937); and, the promoter responsive to the stress hormone abscisic acid (Sheen (1996) Science 274:1900-1902).

In alternative embodiments, M3K-encoding nucleic acids used in methods as provided herein can also be operably linked to plant promoters which are inducible upon exposure to chemicals reagents which can be applied to the plant, such as herbicides or antibiotics. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. Coding sequence can be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324). Using chemically- {e.g., hormone- or pesticide-) induced promoters, i.e., promoter responsive to a chemical which can be applied to the transgenic plant in the field, expression of a polypeptide can be induced at a particular stage of development of the plant.

In alternative embodiments, provided are transgenic plants containing an inducible gene encoding for polypeptides used to practice methods as provided herein whose host range is limited to target plant species, such as corn, rice, barley, wheat, potato or other crops, inducible at any stage of development of the crop.

In alternative embodiments, a tissue-specific plant promoter may drive expression of operably linked sequences in tissues other than the target tissue. In alternative embodiments, a tissue-specific promoter that drives expression preferentially in the target tissue or cell type, but may also lead to some expression in other tissues as well, is used.

In alternative embodiments, proper polypeptide expression may require polyadenylation region at the 3′-end of the coding region. The polyadenylation region can be derived from the natural gene, from a variety of other plant (or animal or other) genes, or from genes in the Agrobacterial T-DNA.

### Engineered Plants Overexpressing M3K-Encoding Nucleic Acids

In alternative embodiments, provided are transgenic plants, plant parts, plant organs or tissue, and seeds comprising a nucleic acid that encodes an M3K δ B3 family enzyme, and expression cassettes or vectors, or a transfected or transformed cell, or transgenic plant comprising or having contained therein an M3K δ B3 family enzyme-encoding nucleic acid. Also provided are plant products, e.g., seeds, leaves, extracts and the like, comprising an M3K δ B3 family enzyme-encoding nucleic acid.

In alternative embodiments, the transgenic plant can be dicotyledonous (a dicot) or monocotyledonous (a monocot). Also provided are methods of making and using these transgenic plants and seeds. The engineered transgenic plant or plant cell over-expressing an M3K δ B3 family polypeptide may be constructed in accordance with any method known in the art. See, for example, U.S. Pat. No. 6,309,872.

Nucleic acids and expression constructs used to practice methods as provided herein can be introduced into a plant cell by any means. For example, nucleic acids or expression constructs can be introduced into the genome of a desired plant host, or, the nucleic acids or expression constructs can be episomes. Introduction into the genome of a desired plant can be such that the host's a CO2Sen protein production is regulated by endogenous transcriptional or translational control elements, or by a heterologous promoter, e.g., a promoter used to drive expression of an M3K δ B3 family enzyme-expressing nucleic acid.

Also provided are engineered plants where insertion of gene sequence into the genome by, e.g., homologous recombination, inserts an M3K δ B3 family polypeptide-encoding nucleic acid sequence.

The nucleic acids practice methods as provided herein can be expressed in or inserted in any plant, plant part, plant cell or seed.

Transgenic plants or a plant or plant cell comprising a nucleic acid used to practice methods as provided herein (e.g., a transfected, infected or transformed cell) can be dicotyledonous or monocotyledonous. Examples of monocots comprising an M3K δ B3 family enzyme-expressing nucleic acid, e.g., as monocot transgenic plants as provided herein, are grasses, such as meadow grass (blue grass, Poa), forage grass such as festuca, lolium, temperate grass, such as Agrostis, and cereals, e.g., wheat, oats, rye, barley, rice, sorghum, and maize (corn). Examples of dicots comprising an M3K δ B3 family enzyme-expressing nucleic acid, e.g., as dicot transgenic plants as provided herein, are tobacco, legumes, such as lupins, potato, sugar beet, pea, bean and soybean, and cruciferous plants (family Brassicaceae), such as cauliflower, rape seed, and the closely related model organism Arabidopsis thaliana. Thus, plant or plant cell comprising an M3K δ B3 family enzyme-expressing nucleic acid, including the transgenic plants and seeds as provided herein, include a broad range of plants, including, but not limited to, species from the genera Anacardium, Arachis, Asparagus, Atropa, Avena, Brassica, Citrus, Citrullus, Capsicum, Carthamus, Cocos, Cojfea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solarium, Sorghum, Theobromus, Trigonella, Triticum, Vicia, Vitis, Vigna, and Zea.

The nucleic acids used to practice methods as provided herein can be expressed in or inserted in any plant cell, organ, seed or tissue, including differentiated and undifferentiated tissues or plants, including but not limited to roots, stems, shoots, cotyledons, epicotyl, hypocotyl, leaves, pollen, seeds, tumor tissue and various forms of cells in culture such as single cells, protoplast, embryos, and callus tissue. The plant tissue may be in plants or in organ, tissue or cell culture.

Transgenic plants In alternative embodiments, provided are transgenic plants, plant cells, organs, seeds or tissues, comprising and expressing the nucleic acids used to practice methods as provided herein, including M3K δ B3 family-expressing genes; for example, provided are plants, e.g., transgenic plants, plant cells, organs, seeds or tissues that show improved growth under limiting water conditions; thus, provided are drought-tolerant plants, plant cells, organs, seeds or tissues (e.g., crops).

A transgenic plant as provided herein can also include the machinery necessary for increasing the expression or activity of an M3K δ B3 family polypeptide encoded by an endogenous gene, for example, by altering the phosphorylation state of the polypeptide to maintain it in an activated state.

Transgenic plants (or plant cells, or plant explants, or plant tissues) over-expressing M3K δ B3 family polypeptide can be produced by a variety of well-established techniques as described above.

Following construction of a vector, most typically an expression cassette, including a polynucleotide, e.g., encoding a transcription factor or transcription factor homolog, standard techniques can be used to introduce the M3K δ B3 family polynucleotide into a plant, a plant cell, a plant explant or a plant tissue of interest. In one aspect the plant cell, explant or tissue can be regenerated to produce a transgenic plant.

The plant can be any higher plant, including gymnosperms, monocotyledonous and dicotyledonous plants. Suitable protocols are available for Leguminosae (alfalfa, soybean, clover, etc.), Umbelliferae (carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. See protocols described in Ammirato et al., eds., (1984) Handbook of Plant Cell Culture—Crop Species, Macmillan Publ. Co., New York, N. Y.; Shimamoto et al. (1989) Nature 338: 274-276; Fromm et al. (1990) Bio/Technol. 8: 833-839; and Vasil et al. (1990) Bio/Technol. 8: 429-434.

Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and

In alternative embodiments, an Agrobacterium tumefaciens mediated transformation is used. Transformation means introducing a nucleotide sequence into a plant in a manner to cause stable or transient expression of the sequence.

Successful examples of the modification of plant characteristics by transformation with cloned sequences which serve to illustrate the current knowledge in this field of technology, and include for example: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,619,042.

In alternative embodiments, following transformation, plants are selected using a dominant selectable marker incorporated into the transformation vector. Such a marker can confer antibiotic or herbicide resistance on the transformed plants, and selection of transformants can be accomplished by exposing the plants to appropriate concentrations of the antibiotic or herbicide.

In alternative embodiments, after transformed plants are selected and grown to maturity, those plants showing a modified trait (e.g., overexpression of M3K δ B3 family enzyme) are identified. The modified trait can be any of those traits described above. In alternative embodiments, to confirm that the modified trait is due to changes in expression levels or activity of the transgenic polypeptide or polynucleotide can be determined by analyzing mRNA expression using Northern blots, RT-PCR or microarrays, or protein expression using immunoblots or Western blots or gel shift assays.

Nucleic acids and expression constructs can be introduced into a plant cell by any means. For example, nucleic acids or expression constructs can be introduced into the genome of a desired plant host, or, the nucleic acids or expression constructs can be episomes. Introduction into the genome of a desired plant can be such that the host's CO2 sensor production is regulated by endogenous transcriptional or translational control elements.

In alternative embodiments, provided are “knockout plants” where insertion of a gene sequence by, e.g., homologous recombination, can result in M3K over-expression. Means to generate “knockout” plants are well-known in the art, see, e.g., Strepp (1998) Proc Natl. Acad. Sci. USA 95:4368-4373; Miao (1995) Plant J 7:359-365. See discussion on transgenic plants, below.

In alternative embodiments, making transgenic plants or seeds comprises incorporating sequences used to practice methods as provided herein and, in one aspect (optionally), marker genes into a target expression construct (e.g., a plasmid), along with positioning of the promoter and the terminator sequences. This can involve transferring the modified gene into the plant through a suitable method. For example, a construct may be introduced directly into the genomic DNA of the plant cell using techniques such as electroporation and microinjection of plant cell protoplasts, or the constructs can be introduced directly to plant tissue using ballistic methods, such as DNA particle bombardment. For example, see, e.g., Christou (1997) Plant MoI. Biol. 35:197-203; Pawlowski (1996) MoI. Biotechnol. 6:17-30; Klein (1987) Nature 327:70-73; Takumi (1997) Genes Genet. Syst. 72:63-69, discussing use of particle bombardment to introduce transgenes into wheat; and Adam (1997) supra, for use of particle bombardment to introduce YACs into plant cells. For example, Rinehart (1997) supra, used particle bombardment to generate transgenic cotton plants. Apparatus for accelerating particles is described U.S. Pat. No. 5,015,580; and, the commercially available BioRad (Biolistics) PDS-2000 particle acceleration instrument; see also, John, U.S. Pat. No. 5,608,148; and Ellis, U.S. Pat. No. 5,681,730, describing particle-mediated transformation of gymnosperms.

In alternative embodiments, protoplasts can be immobilized and injected with a nucleic acid, e.g., an expression construct. Although plant regeneration from protoplasts is not easy with cereals, plant regeneration is possible in legumes using somatic embryogenesis from protoplast derived callus. Organized tissues can be transformed with naked DNA using gene gun technique, where DNA is coated on tungsten microprojectiles, shot 1/100th the size of cells, which carry the DNA deep into cells and organelles. Transformed tissue is then induced to regenerate, usually by somatic embryogenesis. This technique has been successful in several cereal species including maize and rice.

In alternative embodiments, a third step can involve selection and regeneration of whole plants capable of transmitting the incorporated target gene to the next generation. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee (1987) Ann. Rev. of Plant Phys. 38:467-486. To obtain whole plants from transgenic tissues such as immature embryos, they can be grown under controlled environmental conditions in a series of media containing nutrients and hormones, a process known as tissue culture. Once whole plants are generated and produce seed, evaluation of the progeny begins.

In alternative embodiments, after the expression cassette is stably incorporated in transgenic plants, it can be introduced into other plants by sexual crossing. Any of a number of standard breeding techniques can be used, depending upon the species to be crossed. Since transgenic expression of an M3K δ B3 family enzyme-expressing nucleic acid leads to phenotypic changes, plants comprising the recombinant nucleic acids comprising an M3K δ B3 family enzyme-expressing nucleic acid can be sexually crossed with a second plant to obtain a final product. Thus, a seed containing an M3K δ B3 family enzyme-expressing nucleic acid can be derived from a cross between two transgenic plants as provided herein, or a cross between a plant comprising an M3K δ B3 family enzyme-expressing nucleic acid and another plant. The desired effects (e.g., over-expression of an M3K δ B3 family enzyme) can be enhanced when both parental plants express the polypeptides, e.g., an M3K δ B3 family enzyme. The desired effects can be passed to future plant generations by standard propagation means.

Any of the above aspects and embodiments can be combined with any other aspect or embodiment as disclosed here in the Summary and/or Detailed Description sections.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Exemplary Methods for Overexpressing an M3K δ B3 Family Enzyme in Plants

This example demonstrates that methods as provided herein by increasing Raf-like mitogen-activated protein (MAP) kinase (MAPKK) kinase (M3K) activity (i.e., the expression of an M3K δ B3 family enzyme) in a plant or a tree cell can: enhance drought tolerance of crop plants and trees, enhance salinity of tolerance of plants such as crop plants, enhance early monitoring of drought, salinity and cold stress by plants such as crop plants and trees, enhance stress resistance in plants such as crop plants and trees.

Through a combination of a redundancy-circumventing genetic screen and biochemical analyses, we have identified functionally-redundant Raf-like MAPKK kinases (M3Ks) that activate OST1/SnRK2 kinases. Reactivation of dephosphorylated SnRK2 requires these M3Ks, and ABA-induced OST1/SnRK2.6 activation and S-type anion channel activation requires the presence of M3Ks. M3K knock-out plants show not only reduced sensitivity to ABA but also strongly impaired osmotic stress-induced SnRK2 activation. Our results demonstrate that these Raf-like M3Ks are required for ABA- and osmotic stress-activation of SnRK2 kinases, ensuring robust ABA and osmotic stress signal transduction.

Abiotic stresses, including drought and salinity, trigger a complex osmotic-stress and abscisic acid (ABA) signal transduction network. The core ABA signalling components are snf1-related protein kinase2s (SnRK2s), which are activated by ABA-triggered inhibition of type-2C protein-phosphatases (PP2Cs). SnRK2 kinases are also activated by a rapid, largely unknown, ABA-independent osmotic-stress signalling pathway. Here, through a combination of a redundancy-circumventing genetic screen and biochemical analyses, we have identified functionally-redundant MAPKK-kinases (M3Ks) that are necessary for activation of SnRK2 kinases. These M3Ks phosphorylate a specific SnRK2/OST1 site, which is indispensable for ABA-induced reactivation of PP2C-dephosphorylated SnRK2 kinases. ABA-triggered SnRK2 activation, transcription factor phosphorylation and SLAC1 activation require these M3Ks in vitro and in plants. M3K triple knock-out plants show reduced ABA sensitivity and strongly impaired rapid osmotic-stress-induced SnRK2 activation. These findings demonstrate that this M3K clade is required for ABA- and osmotic-stress-activation of SnRK2 kinases, enabling robust ABA and osmotic stress signal transduction.

Data provided herein identifies a family of MAP kinase kinase kinases (M3Ks) that are essential for reactivation of SnRK2 protein kinases after PP2C dephosphorylation. We show that the OST1/SnRK2.6 protein kinase cannot reactivate itself after dephosphorylation. Three independent reconstitution assays and in planta analyses show the function of these M3Ks in SnRK2 kinase reactivation and ABA signalling. Moreover interestingly, triple M3K knockout mutant analyses show that the identified M3Ks are required for the rapid osmotic stress activation of SnRK2 kinases, in a less-well understood, previously proposed, pathway parallel to ABA signalling.

### Results

Isolation of ABA-Insensitive MAPKK-Kinase amiRNA Mutants

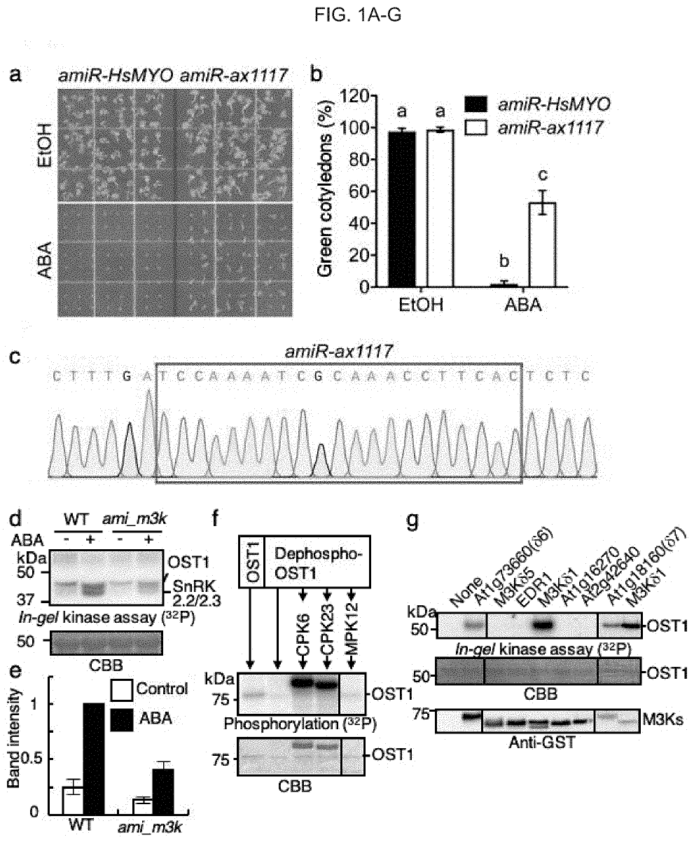

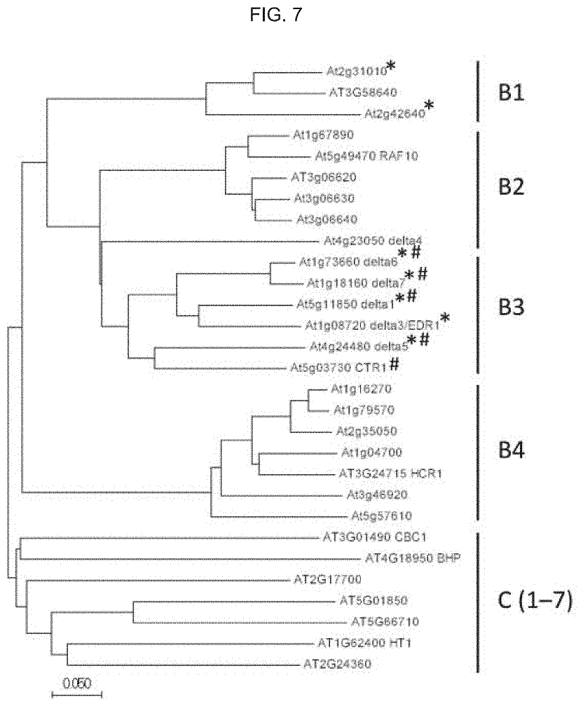

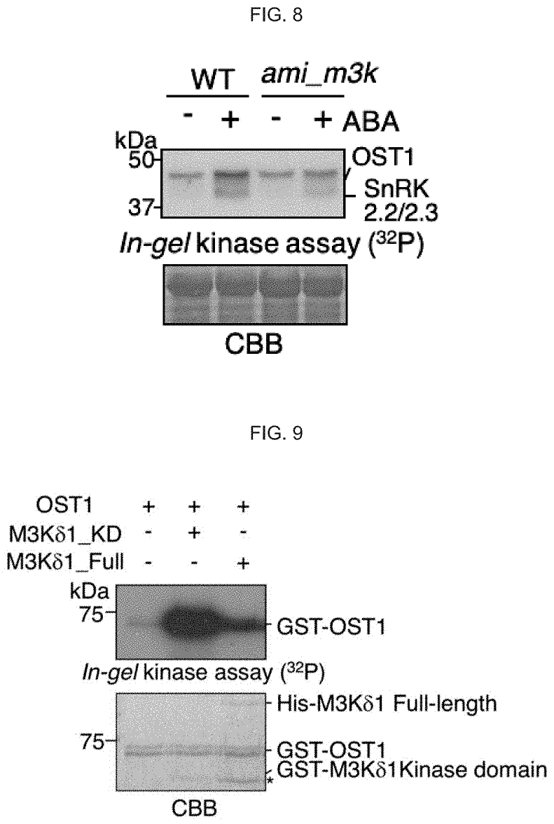

By unbiased forward genetic screening of seeds from over 1,500 independent T2 artificial microRNA (amiRNA)-expressing lines in pools (approximately 45,000 seeds screened) for ABA-insensitive seed germination, we isolated up to ˜290 putative mutants. In secondary screening of the surviving putative mutants in the next (T3) generation, progeny from 25 of the putative mutant plants continued to show a clearly reduced ABA sensitivity, including seeds propagated from three amiR-ax1117-expressing plants (FIG. 1a-c). It is most likely that the three amiRNA-ax1117-expressing plants were the progeny of the same amiRNA-expressing parent line. The amiR-ax1117 is predicted to target five subgroup B Raf-like MAPKK-kinase (M3Ks) genes (FIG. 7). Previously, in a redundancy-circumventing amiRNA pilot screen for impaired ABA inhibition of seed germination in Arabidopsis, we isolated putative mutants, including an M3K amiRNA-expressing line predicted to target seven MAPKK-kinases²¹. These seven putative target M3K genes overlap with four of the above amiR-ax1117 target genes (FIG. 7). Furthermore, in additional genetic screens for ABA-insensitive inhibition of seed germination using more than 2,000 pooled amiRNA-expressing lines (approximately 50,000 seeds screened), we again isolated the previously isolated m3k amiRNA line two more times. The amiR-ax1117 amiRNA and the m3k amiRNA target five and seven overlapping Arabidopsis Raf-like kinase members from subgroup B1 and B3 (FIG. 7). Note that the Arabidopsis genome includes≈80 M3K genes and 22 B family M3K members²². Because SnRK2 protein kinase activation is a key step in ABA signalling, and based on prior findings described further below (FIG. 1f), we investigated ABA-activation of SnRK2 protein kinase activity in seedlings of the m3k amiRNA line by in-gel kinase assays. SnRK2 protein kinases are detected at apparent mobilities of 40 to 44 kDa in in-gel kinase assays^{10,23}. Interestingly, ABA-activation of kinase activities was reduced by 60% (FIG. 1d, e, FIG. 8; n=3 experiments).

ST1/SnRK2.6 Re-Activation after Dephosphorylation

We investigated phosphorylation of purified recombinant GST-tagged OST1/SnRK2.6 protein kinase after dephosphorylation in vitro. To test whether OST1/SnRK2.6 could be re-activated by autophosphorylation, after dephosphorylation, the GST-OST1/SnRK2.6 protein bound on glutathione sepharose 4B resin was incubated with calf intestinal alkaline phosphatase (CIAP), and [γ-³²P]-ATP was added to the reaction after wash out of CIAP. Surprisingly, we found that OST1/SnRK2.6 showed very low autophosphorylation activity even after the protein phosphatase had been removed (FIG. 1f, lane 2; n=3 experiments). Other ABA signalling protein kinases including the calcium-dependent protein kinases CPKδ²⁴ and CPK23²⁵ and the MAP kinase MPK12^{26,27} did not phosphorylate OST1/SnRK2.6 after dephosphorylation (FIG. 1f). Interestingly these results implied that autophosphorylation is not sufficient for OST1/SnRK2.6 re-activation following protein phosphatase exposure and removal. Therefore, another unknown protein kinase may be required for reversible ABA signal transduction.

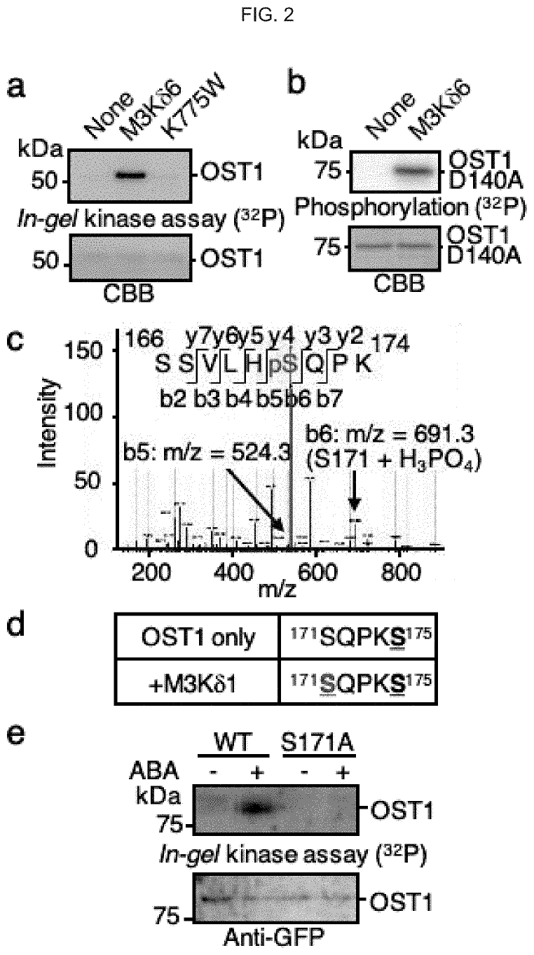

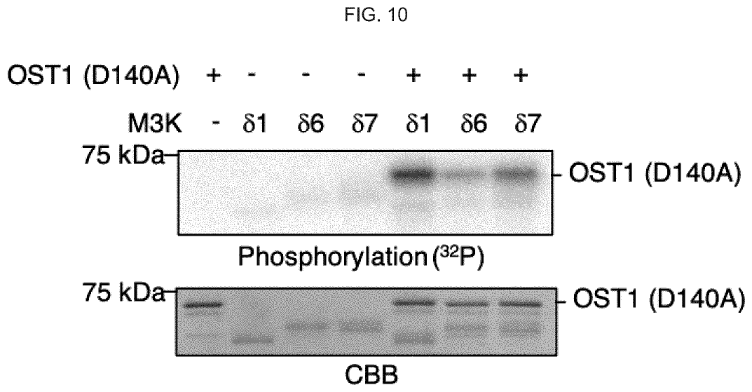

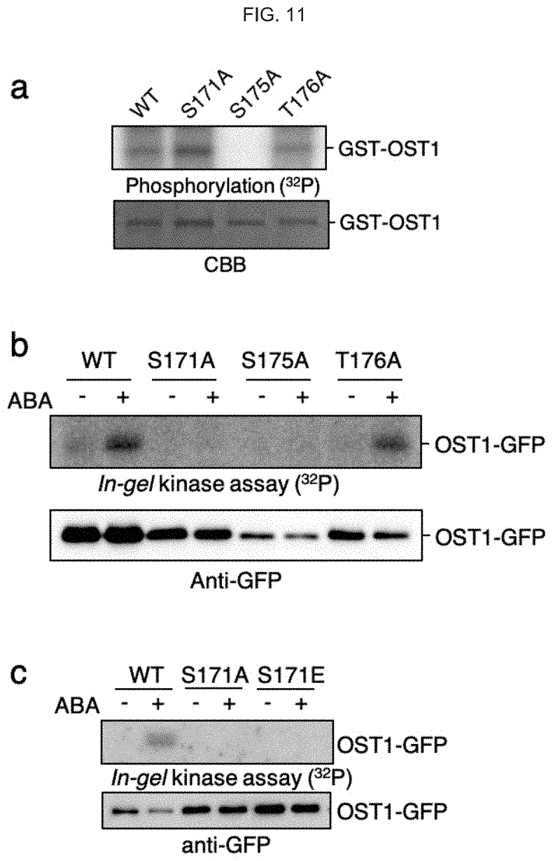

We investigated whether the amiRNA-targeted M3Ks may directly activate OST1/SnRK2.6. In-gel kinase assays were carried out in vitro after incubation of the dephosphorylated His-OST1/SnRK2.6 with GST-tagged recombinant M3K kinase domains and His-tagged full-length M3Ks in the presence of ATP. Notably, three M3Ks from the subgroup B3, named M3Kδ1, 66 and 67, were found to strongly activate OST1/SnRK2.6, whereas the other M3Ks targeted by the corresponding amiRNA did not clearly activate OST1/SnRK2.6 under the imposed conditions in vitro (FIG. 1g and FIG. 9; n=3 experiments). OST1/SnRK2 kinase activation was not induced by an inactive mutant M3K kinase protein, M3Kδ6 (K775W) (FIG. 2a). Moreover, the M3Kδ1, 66 and 67 kinase domains directly phosphorylated the kinase inactive OST1/SnRK2.6 (D140A) mutant isoform (FIG. 2b and FIG. 10). Note that a Physcomitrella patens protein kinase ARK showing similarity to these M3Ks was recently reported to phosphorylate a Physycomitrella SnRK2 kinase²⁸.

M3Kδ1 Phosphorylates a Critical Ser171 for OST1 activation

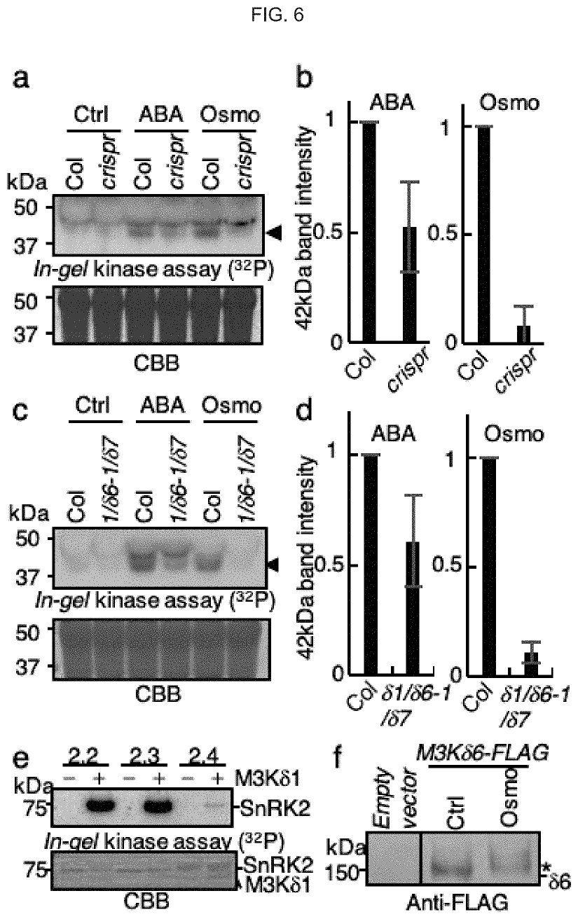

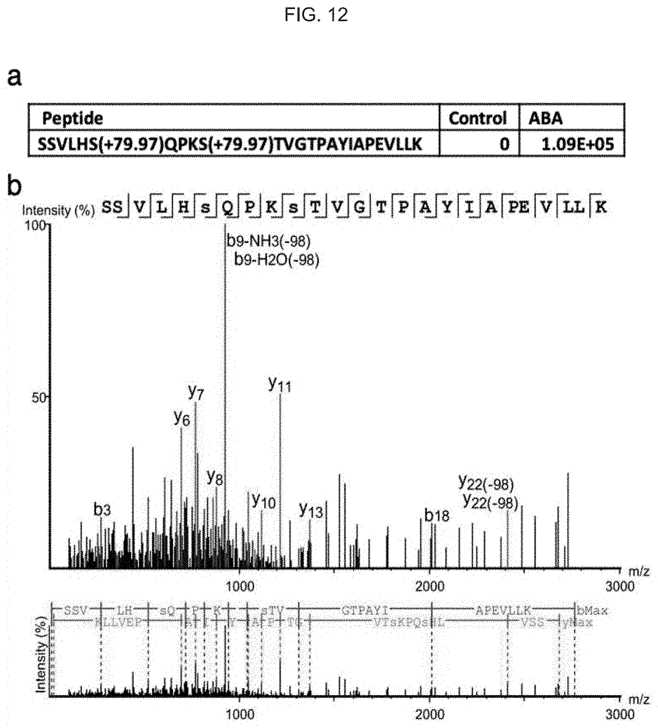

Mass spectrometry analyses revealed that M3Kδ1 phosphorylated the OST1/SnRK2.6 residues Ser171, Ser175 and Thr176 in the OST1 activation loop (FIG. 2c). We next focused on Ser171, because this site has not been found as an OST1/SnRK2.6 autophosphorylation site in vitro¹⁶, consistent with our mass spectrometry analyses of OST1 (FIG. 2c, d). Using Arabidopsis mesophyll cell protoplasts as a transient expression system, consistent with a previous study¹¹, we found that substitution of this OST1/SnRK2.6 Ser171 by an alanine completely abrogated ABA-dependent activation of OST1/SnRK2.6 (FIG. 2e; n=3 experiments). Notably, the OST1-S171A mutation does not disrupt kinase activity in vitro, while another phosphorylation site mutation (S175A) disrupts kinase activity (FIGS. 11a and b). An OST1/SnRK2.6 T176A mutation does not disrupt kinase activity in vitro nor does the T176A mutation affect ABA activation of OST1/SnRK2.6 in vivo (FIGS. 11a and b). These results suggest that Ser171 plays an important role in ABA-activation of OST1/SnRK2.6 in plant cells. A potential phospho-mimic isoform of Ser171, OST1/SnRK2.6 (S171E) has no detectable kinase activity in mesophyll cells (FIG. 11c). This is consistent with a previously reported OST1/SnRK2.6 (S171D) mutant protein¹¹. We further investigated the effect of ABA on phosphorylation of OST1-S171 in mesophyll cells. Ser171 is phosphorylated in plant mesophyll cells in response to ABA (Supplementary FIG. 6)^{10,11}.

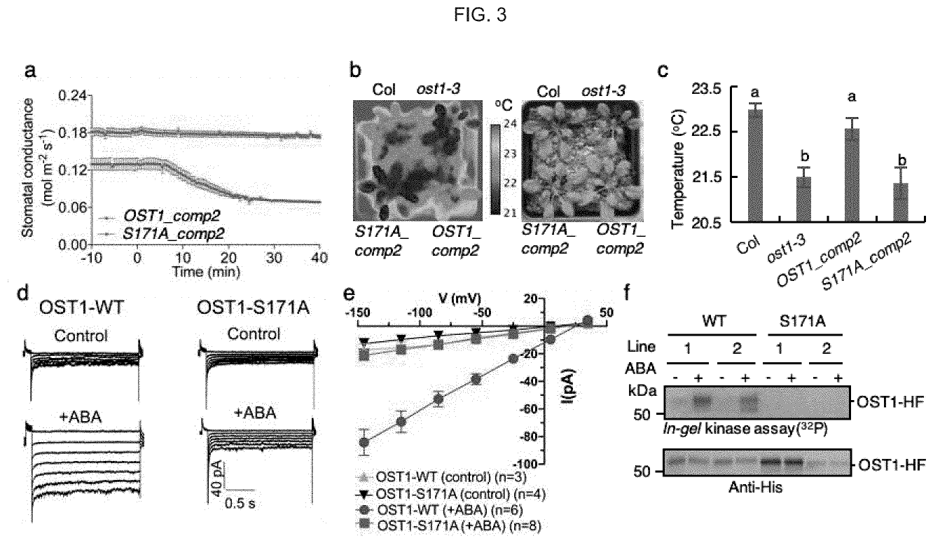

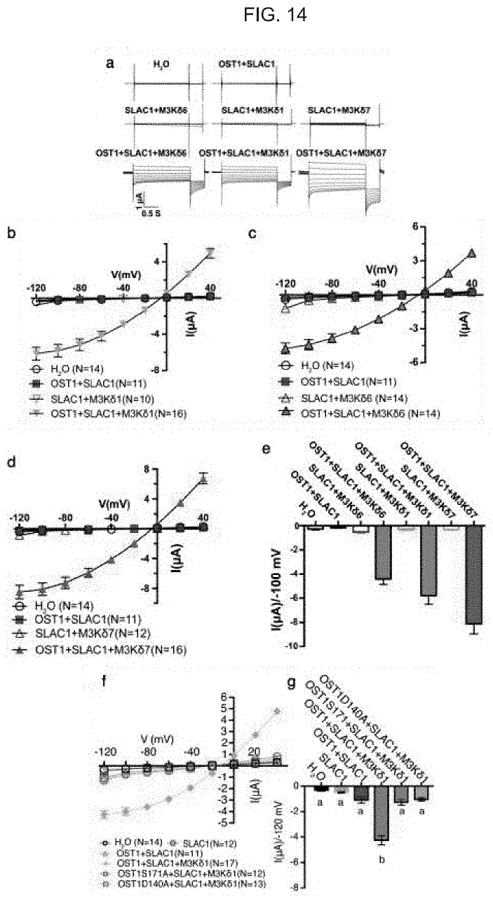

We created transgenic Arabidopsis plants stably expressing OST1-HF (S171A) in the ost1-3 background^{29,30}. Expression of OST1-HF (S171A) did not rescue the ABA-insensitive stomatal conductance response and the low leaf temperature phenotype of the ost1-3 mutant in two independent lines (FIG. 3a-c and FIG. 13). Complementation of ost1-3 with the wildtype OST1-HF isoform restored ABA-induced stomatal closing and warm leaf temperatures (FIG. 3a-c and FIG. 13), together indicating that Ser171 is required for OST1/SnRK2.6 function in stomatal closing (FIG. 3a-c and FIG. 13).

Patch-clamp analyses of the ost1-3 complementation lines showed the essential role of Ser171 for ABA-induced S-type anion channel activation in Arabidopsis guard cells (FIG. 3d, e). We further found that, in contrast to OST1-HF-expressing controls, OST1-HF (S171A) was not activated in Arabidopsis mesophyll cells in response to ABA in these stable homozygous transgenic plant lines (FIG. 3f).

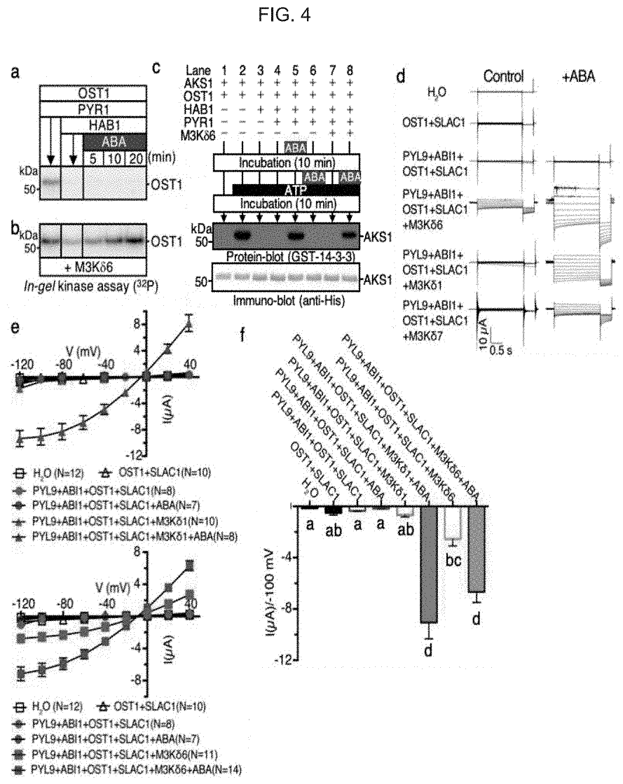

Reconstitution of early ABA signalling with MAPKK-kinases Previous studies have reconstituted ABA-dependent phosphorylation of OST1/SnRK2.6 substrates in vitro using recombinant proteins^{14,18}. Recombinant OST1/SnRK2.6 has many phosphorylated sites and a significant protein kinase activity in vitro¹⁶. However, we find that prior dephosphorylated OST1/SnRK2.6 could unexpectedly not be re-activated by itself (FIG. 1f). We therefore hypothesized that these M3Ks have a role in re-activation of SnRK2 after inactivation by PP2C-mediated dephosphorylation. To test this, we pursued in vitro reconstitution experiments using recombinant proteins PYR1/RCAR11, the HAB1 PP2C, OST1/SnRK2.6 with or without M3Kδ6. In-gel kinase assays clearly showed that when HAB1-dependent OST1/SnRK2.6 dephosphorylation preceded ABA application, PYR1/RCAR11, HAB1 and OST1/SnRK2.6 could not recover OST1/SnRK2.6 activation (FIG. 4a; n>3 experiments). Moreover, OST1/SnRK2.6 was no longer activated even after ABA treatment (FIG. 4a; n>3 experiments). However, the OST1/SnRK2.6 kinase was clearly re-activated in response to ABA when M3Kδ6 was added to these reactions (FIG. 4b; n>3 experiments). Consistent with these findings, in vitro reconstitution of ABA-dependent AKS1 transcription factor phosphorylation by OST1/SnRK2.6¹⁸ was not observed when ABA was added after OST1/SnRK2.6 had been initially dephosphorylated by the PP2C HAB1 for 10 min (FIG. 4c, compare lanes 5, 6). Addition of M3Kδ6 restored ABA-induced His-AKS1 phosphorylation (FIG. 4c, compare lanes 7, 8).

### Reconstitution of ABA Activation of SLAC1 Requires M3Ks

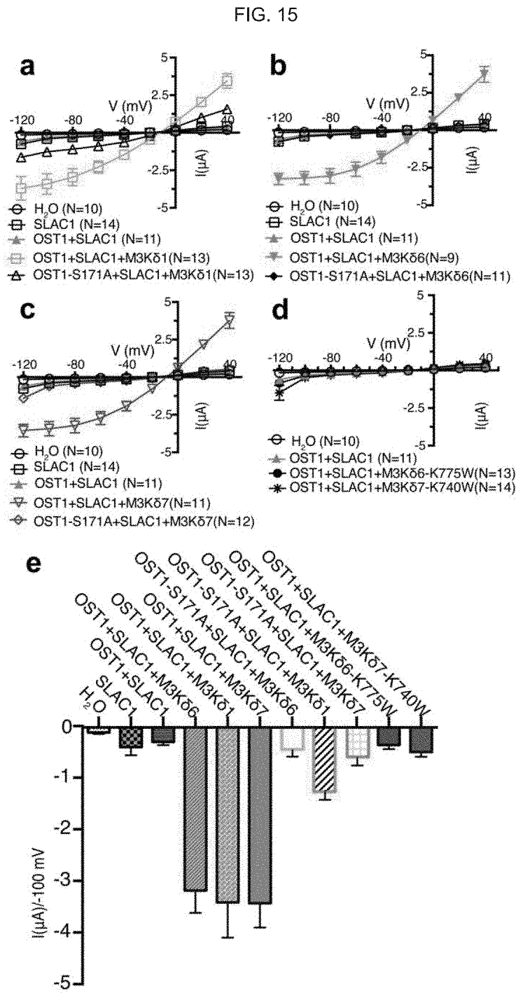

OST1/SnRK2.6-mediates activation of the S-type anion channel SLAC1 in Xenopus oocytes^{12,13}, and ABA-induced SLAC1 activation was reconstituted in oocytes¹⁷. These results strongly depended on artificial BiFC tags that force interaction of the SLAC1 channel with OST1/SnRK2.6 proteins^{12,17}, indicating that the BiFC tag might cause an unknown artificial effect. When expressing SLAC1 and OST1/SnRK2.6 proteins without any tag in oocyte experiments in the present study, SLAC1 was not significantly activated (FIG. 14a-e). We found that SLAC1 was strongly activated when small amounts of M3Kδ1, M3Kδ6 or M3Kδ7 cRNA were co-injected with OST1 into oocytes (FIG. 14a-e; ratio of [M3K] to [OST1] cRNA=1 to 10). However, the M3Ks did not activate SLAC1 in the absence of OST1/SnRK2.6 (FIG. 14a-e), even when the injected M3K to SLAC1 cRNA concentration ratio was 1 to 1. Furthermore, kinase inactive OST1/SnRK2.6 (D140A) does not activate SLAC1 in the presence of M3Kδ1 (FIG. 14f, g).

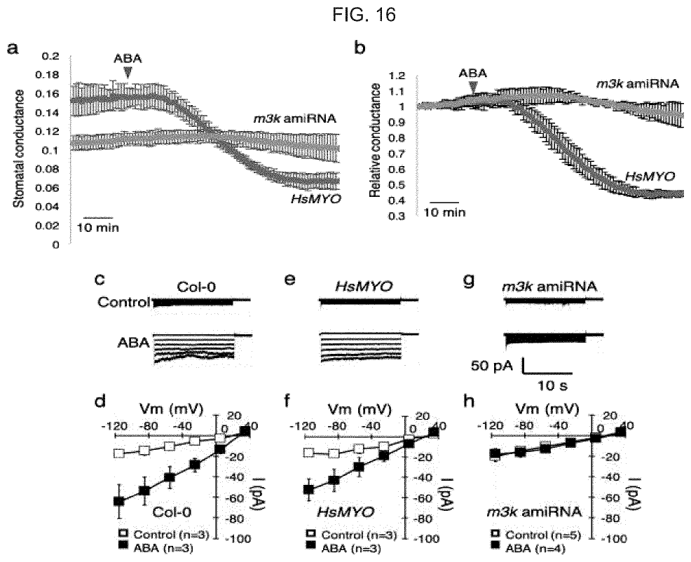

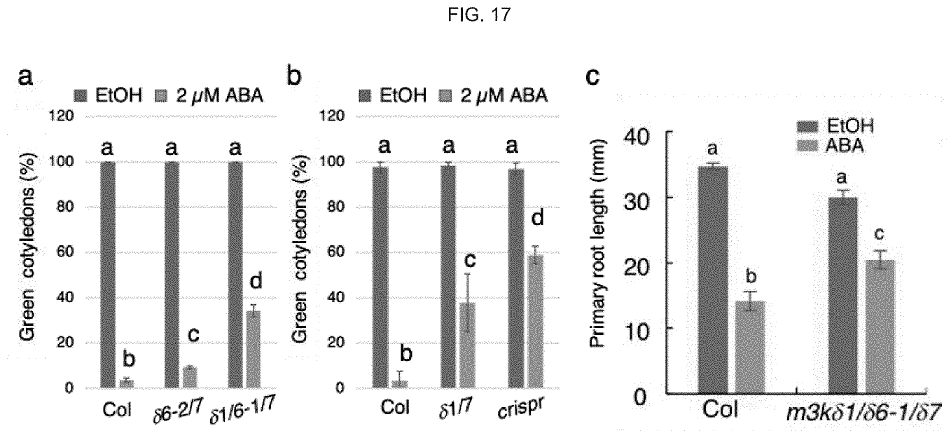

In additional experiments, we co-injected cRNA for the ABA receptor PYL9/RCAR1, together with the ABI1 PP2C, OST1 SnRK2.6, SLAC1 and M3Ks into oocytes, to test whether ABA-dependent SLAC1 anion channel activation could be reconstituted with these components. ABA could activate SLAC1 in oocytes only in the presence of low concentrations of either M3Kδ1, M3Kδ6 or M3Kδ7 mRNAs (FIG. 4d-f). Moreover, inactive M3K kinase mutant isoforms and inactive OST1 (S171A) disrupted reconstitution of SLAC1 activation (FIG. 15). As SLAC1 plays an important role in ABA-induced stomatal closing, gas exchange experiments were pursued. m3k amiRNA plants show a reduced steady-state stomatal conductance and an ABA insensitivity in stomatal closure (FIG. 16a, b).

The reduced steady-state stomatal conductance in the m3k amiRNA line indicates additional effects of this artificial microRNA and/or compensatory effects of impaired stomatal closing response mutants^{31,32}. Higher order mutant combinations will be required to investigate this hypothesis. Based on the lower steady-state stomatal conductance, the impaired response to ABA (FIG. 16a, b) and findings showing that ABA activation of S-type anion channels is an important mechanism for ABA-induced stomatal closing^{24,33}, we investigated ABA activation of S-type anion channels in guard cells. ABA (10 μM) caused typical ABA activation of S-type anion currents in guard cells of the wildtype (Col-0) and the HsMYO control line (FIG. 16c-f). In contrast, ABA activation of S-type anion channels was impaired in guard cells of the m3k amiRNA line (FIG. 16g, h). ABA signalling reconstitution (FIG. 4) and guard cell anion channel regulation analyses (FIG. 16c-h) together suggest that the identified M3Ks provide a missing component of the early ABA signalling module.

### Higher Order M3K Mutants Show ABA Insensitive Phenotypes

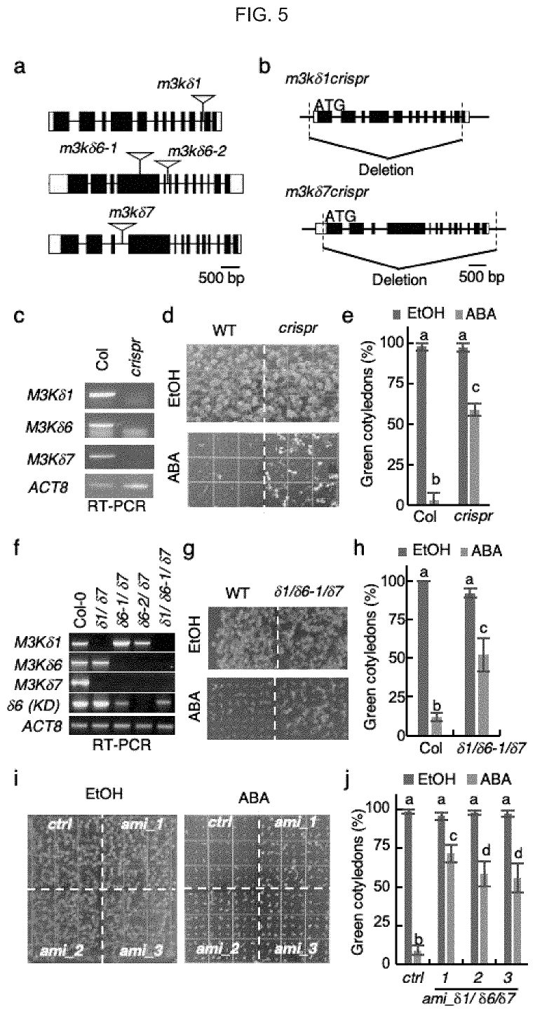

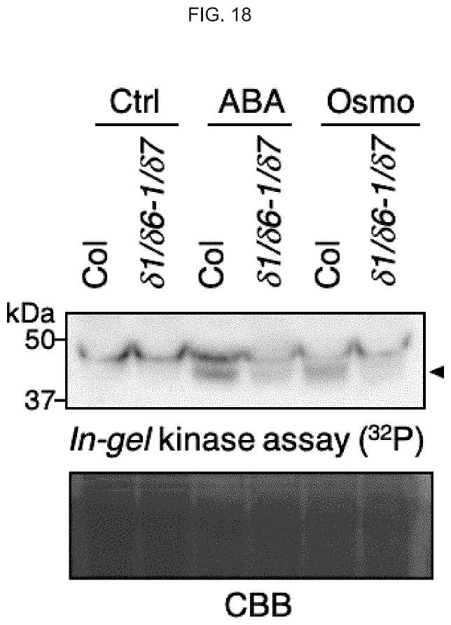

We isolated T-DNA insertion mutants (m3kδ1 (SALK_048985), m3kδ6-1 (SALK_004541), m3kδ6-2 (SALK_001982) and m3kδ7 (SALK_082710)) (FIG. 5a). We also deleted large fragments of the M3Kδ1 or M3Kδ7 genes by CRISPR-Cas9 in the m3kδ6-2 T-DNA knock-out background (FIG. 5b), and a triple knock-out mutant (m3kδ1crispr m3kδ6-2 m3kδ7crispr) was generated by crossing these lines (FIG. 5c) to analyze the physiological functions of these M3K genes. The m3kδ1crispr m3kδ6-2 m3kδ7crispr triple mutant showed a reduced ABA sensitivity phenotype in green cotyledon emergence from seeds (FIG. 5d,e). The double mutants m3kδ1 m3kδ7 and m3kδ6-2 m3kδ7 showed weaker ABA insensitive phenotypes than the triple mutants (FIG. 17a, b). Also, m3kδ1/δ6-1/δ7 mutant seedlings showed a reduced ABA sensitivity in inhibition of primary root elongation on ½MS plates supplemented with ABA (FIG. 17c).

We confirmed knock-out of full-length expression of M3Kδ1 and M3Kδ7 in the T-DNA lines, while there was partial expression of the kinase domain of M3Kδ6 in the m3kδ6-1 line (FIG. 5f). Seed germination analyses showed reduced ABA sensitivity in the m3kδ1 m3kδ6-1 m3kδ7 T-DNA insertion triple mutants (FIG. 5g, h). Another T-DNA allele for M3Kδ6 for which the full length and kinase domain transcripts could not be amplified (FIG. 5a; m3kδ6-2) was considered. However, we could not isolate a viable m3kδ1 m3kδ6-2 m3kδ7 triple mutant, possibly due to homozygous lethality, likely linked to an unknown second site mutation. Because the partial expression of the M3Kδ6 kinase domain fragment was detected in the m3kδ6-1 mutant (FIG. 5f), this kinase fragment may weaken the phenotypic effect.

To further test the function of these M3Ks, we created amiRNA lines predicted to target only the triple combination of M3Kδ1, M3Kδ6 and M3Kδ7 and found that three independent amiRNA lines showed ABA-insensitivities in seed germination (FIG. 5i, j). Together these results support that these M3Ks have a function in ABA responses.

### ABA- and Osmotic Stress-SnRK2 Activations Require M3Ks

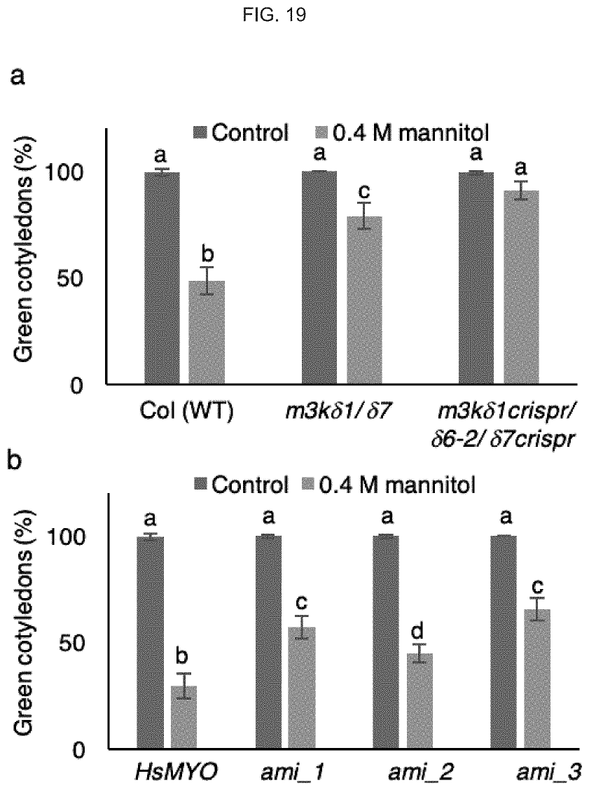

In-gel kinase assays showed that ABA-induced activation of SnRK2 kinase in the m3kδ1crispr m3kδ6-2 m3kδ7crispr triple was slightly less strong than in wild type plants (FIG. 6a, b; n=4 experiments). We further found a slightly reduced ABA activation of SnRK2 kinase activity in the T-DNA insertion m3kδ1 m3kδ6-1 m3kδ7 triple mutant compared to wildtype controls (FIG. 6c, d, FIG. 18; n=4 experiments), similar to the m3kδ1crispr m3kδ6-2 m3kδ7crispr triple knock out mutant allele findings. Osmotic stress is known to rapidly activate OST1/SnRK2.6 independent of ABA signalling²⁰. Interestingly, we found that 15 min osmotic stress-induced SnRK2 activation was strongly impaired in these two independent m3k triple mutant alleles, and this impairment was stronger than that in response to ABA application (FIG. 6a-d, FIG. 18; n=4 experiments per allele).

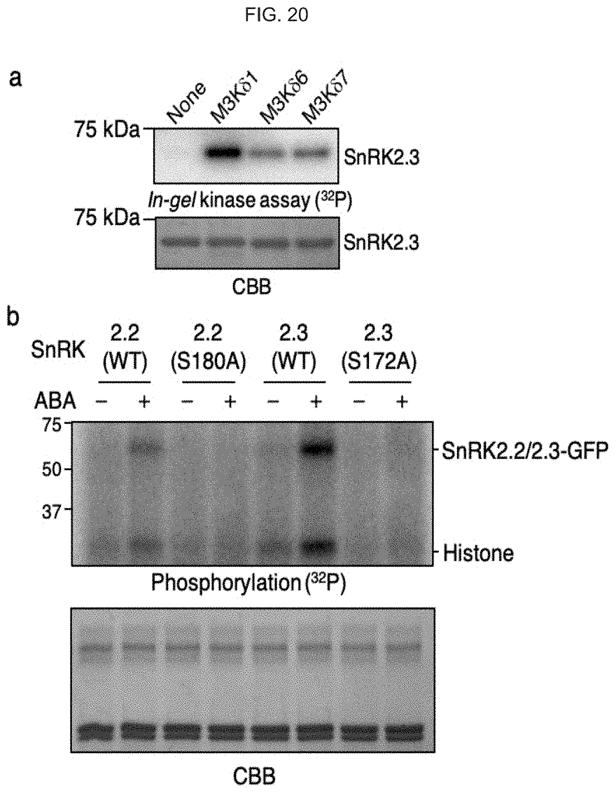

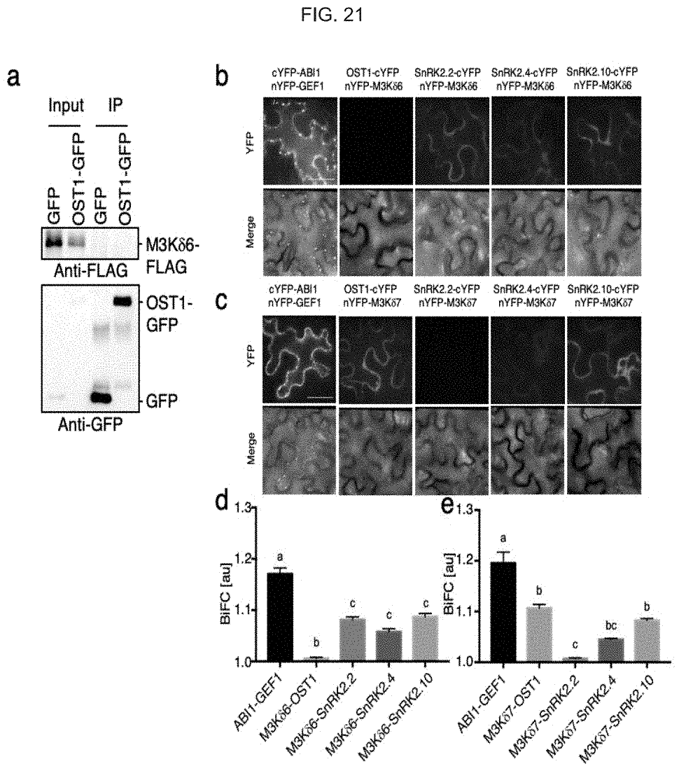

In-gel kinase assays suggest that the M3Ks have a major role in osmotic stress signalling in Arabidopsis (FIG. 6a-d). We therefore investigated osmotic-stress responses of the m3k double and triple mutants and the m3k amiRNA lines, and found that they showed reduced sensitivity to osmotic-stress in seed germination assays (FIG. 19). SnRK2 gene functions are highly redundant in mediating osmotic stress resistance34. At least nine members out of the ten Arabidopsis SnRK2 proteins are activated by osmotic stress through unknown mechanisms, while three members (SnRK2.2/2.3/2.6) are major ABA-activated SnRK2s^{19,23}. In vitro in-gel kinase assays showed that M3Kδ1 strongly activated SnRK2.2 and 2.3 (FIG. 6e) as well as OST1/SnRK2.6 (FIG. 1g). SnRK2.3 was also activated by M3Kδ6 and M3Kδ7 (FIG. 20a). We also found that SnRK2.2 (S180A) and SnRK2.3 (S172A), which have a mutation corresponding to OST1/SnRK2.6 (S171A), are not activated by ABA in mesophyll cell protoplasts in contrast to WT SnRK2.2 and WT SnRK2.3 (FIG. 20b). M3Kδ1 also activated SnRK2.4 kinase in vitro that is known to be activated by osmotic stress¹⁹.

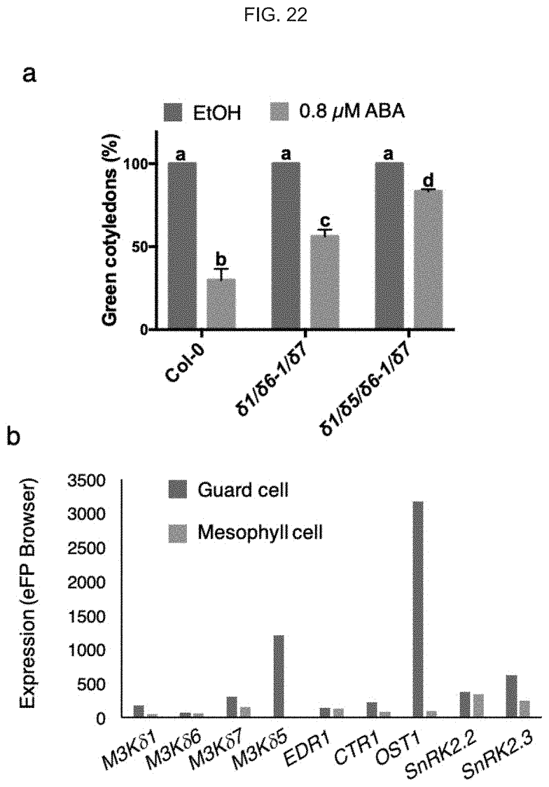

Co-immunoprecipitation of M3Kδ6- and OST1/SnRK2.6-expressed in mesophyll cell protoplasts did not show a clear interaction (FIG. 21a). Protein kinase interactions are often transient and do not show co-immunoprecipitation with their targets³⁵. BiFC analyses can detect transient interactions in plant cells. Quantitative BiFC experiments provide evidence that M3Kδ6 and M3Kδ7 bind to OST1/SnRK2.6, SnRK2.2, SnRK2.4 and SnRK2.10 in plant cells with different efficiencies (FIG. 21b-e). We further observed that the M3Kδ6-FLAG protein band in SDS-PAGE gels was slightly shifted in response to 15 min osmotic stress treatment in mesophyll cell protoplasts, suggesting an osmotic stress-dependent post-translational modification of M3Kδ6 (FIG. 6f, n=3).

### Discussion

In the present study, a combination of genetic screening for functional redundancy in abscisic acid responsiveness and multiple biochemical and signal transduction analyses in vitro and in planta have identified and characterized members of the Raf-like MAPKK-kinase δ B3 family that are required for full activation of SnRK2 protein kinases in abscisic acid signal transduction in vitro (FIG. 1g and FIG. 4a-c), in a reconstitution system (FIG. 4d-f) and in planta (FIGS. 1a-e, 5d-j and 6a-d). Triple mutants in the M3Ks AtM3Kδ1, M3Kδ6 and M3Kδ7 show impaired ABA- and osmotic stress-responses. As the Arabidopsis genome includes 80 MAPKK-kinases²², of which 22 MAPKK-kinases are in the B subgroup, it is conceivable that additional members of this family contribute to ABA responses and that higher order mutants will cause enhanced ABA insensitivity. Previous studies suggest that other MAPKK-kinases, than those identified here, are involved in aspects of ABA signalling through a MAP3K-MAP2K-MAPK cascade³⁶⁻³⁸ or through unknown pathways^{39,40}.

Dephosphorylation of the OST1/SnRK2.6 kinase was unexpectedly found not to result in OST1/SnRK2.6 re-activation by SnRK2 auto-phosphorylation alone. The identified M3Kδs, but not other analyzed CPK and MPK12 protein kinases that function in ABA signalling²⁴⁻²⁷, were found to be required for re-activation of OST1/SnRK2.6. Moreover, the M3Kδ1 kinase greatly enhances the activities of other ABA signalling protein kinases SnRK2.2 and SnRK2.3 (FIG. 6e). Furthermore, M3Kδs re-activate OST1/SnRK2.6 through phosphorylation of Ser171 in OST1/SnRK2.6. The Ser171 residue in OST1/SnRK2.6 is essential for ABA responses in planta (FIG. 3 and FIG. 13), but OST1/SnRK2.6 cannot auto-phosphorylate this Ser-171 residue (FIG. 2)^{11,16}. These data point to the model that the M3Kδs identified here are essential for SnRK2 kinase re-activation and thus robust ABA responses in plants. Higher order M3K mutants and further experiments will be needed to investigate M3K-dependent Ser171 phosphorylation of OST1/SnRK2.6 in planta.

A previous proof-of-concept screen using artificial microRNAs that target multiple homologous genes isolated a plant predicted to target seven M3Ks of the B-family²¹. A Physcomitrella single gene encoding a M3K, ARK, was also identified which functions in SnRK2 activation²⁸. Recent studies show that ARK kinase is required for Physcomitrella ABA and drought stress responses including phosphorylation of transcription factors through SnRK2 kinases^{41,42}. Here, in forward genetic screening we have isolated amiRNA expressing lines that target M3K members of the B family (FIG. 1a-c and FIG. 7). In the present study, we show that for prior dephosphorylated SnRK2 kinases, we could robustly reconstitute ABA-activation of OST1/SnRK2.6 and the SLAC1 anion channel only in the presence of M3Kδs in vitro and in Xenopus oocytes (FIG. 4). The present experiments reveal that auto-phosphorylation cannot alone reactivate the SnRK2 kinases. These data suggest that these M3Ks are a missing component of the early ABA signalling module in plants.

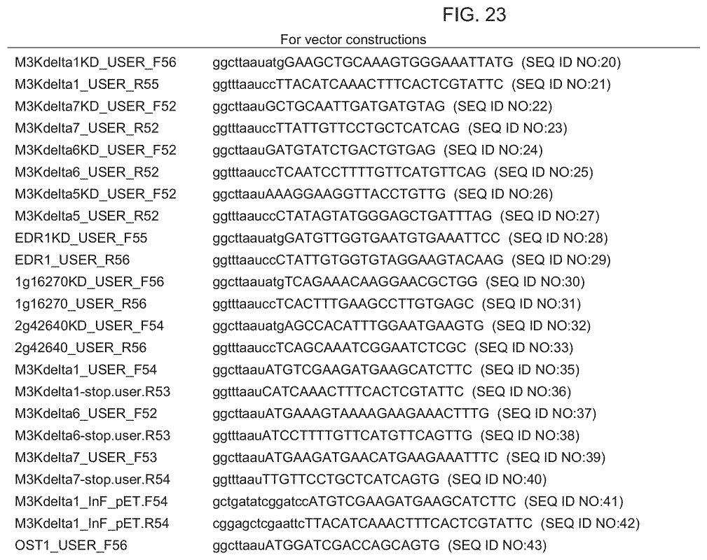

Osmotic stress is known to rapidly activate SnRK2 protein kinases^{20,34,43}. Rapid osmotic stress signalling includes a prominent ABA-independent pathway that leads to activation of transcription factors^{44,45}. However, the upstream osmotic stress signalling mechanisms remain incompletely understood. Recent studies suggest that PP2Cs involved in ABA signalling dephosphorylate SnRK2.4⁴⁶⁻⁴⁸. The M3K ARK is required for osmotic stress tolerance in Physcomitrella^{28,42}. Interestingly, the identified M3Kδs play a critical role in the rapid osmotic stress activation of SnRK2 protein kinases (FIG. 6a-d). In m3k triple mutants, 15 min short term osmotic stress activation of SnRK2 is greatly impaired in planta. This impairment in rapid osmotic stress activation of SnRK2 protein kinases is prominent in the investigated m3kδ1/δ6/δ7 triple mutant alleles, in contrast to that of ABA activation of SnRK2 kinases (FIG. 6a-d). Further research will be needed to determine whether higher order m3k mutants further impair the ABA response. To start testing this hypothesis, we created m3kδ/δ5/δ6-1/δ7 quadruple mutant plants and found that they show a stronger ABA-insensitive phenotype in seed germination than the triple mutant (FIG. 22a). Triple mutant plants, which include the weak allele m3kδ6-1 (m3kδ1/δ6-1/δ7), did not show a clear phenotype in ABA-induced stomatal closing using a robust method of gas exchange analyses. The public eFP Browser shows a prominent guard cell expression of M3Kδ5 (FIG. 22b). M3Kδ5 is targeted by the m3k amiRNA (FIG. 7), which shows an ABA-insensitive stomatal closing (FIG. 16a, b) and impairs ABA activation of S-type anion channels (FIG. 16c-h). Higher order mutants will be required to further investigate M3K functions in ABA-induced stomatal closing. The requirement of M3Kδs for the rapid osmotic stress response suggests that these M3Kδs also mediate osmotic stress signal transduction before the slower onset of ABA concentration increase 4 to 6 hours after exposure to osmotic stress⁴⁹. These findings are consistent with previous observations of an ABA-independent osmotic stress-triggered SnRK2 signal transduction pathway^{20,43,50}. The present study points to a model in which the identified M3K6 protein kinases may act as a convergence point of rapid osmotic stress signalling and prolonged abscisic acid signal transduction.

Osmotic and salt stresses induce a rapid cytosolic Ca²⁺ increase⁵¹⁻⁵⁴. An ABA-independent osmotic stress signalling pathway has been characterized that triggers rapid gene expression^{44,55}. Recent research shows that the Arabidopsis NGATHA1 transcription factor mediates the ensuing drought stress-induced ABA accumulation through enhanced expression of the ABA biosynthesis NINE-CIS-EPOXYCAROTENOID DIOXYGENASE, NCED3⁵⁶. Gel shift assays indicate that osmotic stress causes a rapid post-translational modification of M3Kδ6 (FIG. 6f). Our results reveal a key component by which plants respond initially to osmotic stress before measurable stress-induced ABA concentration increases in roots. Furthermore, interestingly, m3k amiRNA lines impair robust ABA activation of SnRK2 kinases in planta. Further research will be required to elucidate the presently unknown mechanisms between osmotic stress sensing and M3Kδ-dependent activation of SnRK2 protein kinases.

### Methods

### Genetic Screening for ABA Response Mutants

Using amiRNA libraries²¹, we screened amiRNA lines for ABA-insensitive seed germination phenotypes using ½ MS plate supplemented with 2 μM ABA⁵⁷. The underlying amiRNA sequences were identified from genomic DNA by PCR and sequencing (m3k amiRNA: 5′-TTGGAGCCATCCATTCAGCCG-3′ (SEQ ID NO:1), amiR-ax1117: 5′-TCCAAAATCGCAAACCTTCAC-3′) (SEQ ID NO:2). We used an amiRNA line targeting human myosin 2 gene (HsMYO2) as a control.

### In Vitro Dephosphorylation and Phosphorylation Assays

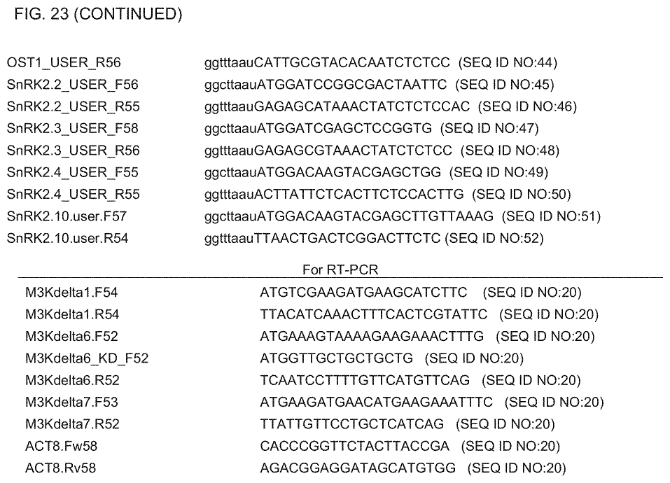

10 μg GST-OST1/SnRK2.6 proteins were bound to glutathione sepharose 4B beads and incubated with 30 U CIAP for 2 hr at room temperature. The beads were washed with T-TBS (50 mM Tris-HCl pH7.5, 150 mM NaCl, 0.05% Tween-20) three times, and GST-OST1 protein was eluted with 30 μL elution buffer (50 mM Tris-HCl pH 8.0, 10 mM reduced glutathione). 5 μL GST-OST1 solution was added in phosphorylation buffer [50 mM Tris-HCl pH 7.5, 10 mM MgCl₂, 2 μM free Ca²⁺ buffered by 1 mM EGTA and CaCl₂ (https://somapp.ucdmc.ucdavis.edu/pharmacology/bers/maxchelator/CaMgATPEGTA-NIST.htm), 0.1% Triton X-100, and 1 mM DTT] with or without 1 μg of the protein kinases CPKδ, CPK23, MPK12 or 0.1 μg of the indicated MAPKK kinases (M3Ks). The phosphorylation reactions were started by addition of 200 μM ATP and 1 ρCi [γ-³²P]ATP. After 60 min incubation at room temperature, these reactions were stopped by addition of SDS-PAGE loading buffer. Note that the mobilities of recombinant and transgenic proteins in the present study depend on the linked tags. For example, the OST1/SnRK2.6 6×His-tag also includes sequences including thrombin and enterokinase cleavage sites and restriction enzyme sites in the pET-30a(+) vector used for E. coli expression of OST1/SnRK2.6 in FIGS. 1g, 4a and b. Primer sequences used for cloning in this study are provided in FIG. 23.

### In-Gel Kinase Assays

15-20 Arabidopsis seedlings (7-9-day-old) grown on ½ MS plates were treated with 10 μM ABA or 0.3 M mannitol for 15 min at room temperature and grinded with a pestle and mortar in 400 μL extraction buffer (50 mM MOPS-KOH pH 7.5, 100 mM NaCl, 2.5 mM EDTA, 10 mM NaF, 2 mM dithiothreitol, 1 mM phenylmethylsulfonyl fluoride, 10 μM leupeptin) on ice. After 10 min centrifugation at 13,000 g, the supernatants were transferred to new tubes, and proteins were precipitated by acetone precipitation. Proteins were dissolved in SDS-PAGE loading buffer and separated in 9% acrylamide gels. In-gel kinase assays were performed as described previously⁵⁸. In brief, gels were incubated in washing buffer (25 mM Tris-HCl pH 8.0, 0.5 mM DTT, 0.1 mM Na₃VO₄, 5 mM NaF, 0.5 mg ml⁻¹ BSA, and 0.1% Triton X-100) for 30 min three times and in renaturation buffer (25 mM Tris-HCl pH 8.0, 1 mM DTT, 0.1 mM Na₃VO₄, and 5 mM NaF) for 30 min once. Gels were further incubated in renaturation buffer at 4° C. overnight followed by further incubation in reaction buffer (50 mM Tris-HCl pH 7.5, 10 mM MgCl₂, 2 mM DTT, and 1 mM EGTA) for 30 min. Phosphorylation reactions were carried out in reaction buffer with 50 μCi [γ-³²P]-ATP for 60 min at room temperature. Gels were washed in 5% trichloroacetic acid and 1% phosphoric acid four times for 30 min each. Storage phosphor screens or X-ray films were used for detection.

### In Vitro Reconstitution of ABA Signalling

0.43 μmol His-OST1/SnRK2.6, 0.17 μmol His-PYR1/RCAR11 and 0.06 μmol GST-M3Kδ6 kinase domain were incubated in 200 μL phosphorylation buffer (50 mM Tris-HCl pH 7.5, 10 mM MgCl₂, 0.1% Triton X-100, and 1 mM DTT) with 200 μM ATP for 10 min, and 20 μL solution was transferred to a new tube and 10 μL 3×SDS-PAGE loading buffer was added to stop the reaction. Then, 0.01 μmol His-HAB1 was added to the reaction solution, and 20 μL solution were transferred to a new tube to stop the reaction by addition of 10 μL 3×SDS-PAGE loading buffer after 10 min incubation. 50 μM ABA was added to the reaction and 20 μL reactions were transferred to new tubes to stop the reaction after 5, 10 or 30 min incubation. Proteins were separated by SDS-PAGE, and OST1/SnRK2.6 activity was detected by in-gel kinase assays.

### Identification of OST1/SnRK2.6 Phosphorylation Sites

30 μg GST-OST1/SnRK2.6(D140A) and 2.5 μg GST-M3Kδ1 kinase domain were incubated in phosphorylation buffer (50 mM Tris-HCl pH 7.5, 10 mM MgCl₂, 0.1% Triton X-100, and 1 mM DTT) with 1 mM ATP for 2 hr at room temperature. Proteins were precipitated by acetone precipitation and dissolved in SDS-PAGE loading buffer. After SDS-PAGE and CBB staining, protein bands of GST-OST1/SnRK2.6(D140A) were excised and analyzed by LC-MS/MS¹⁷. For in vivo Ser-171 phosphorylation, OST1/SnRK2.6-GFP was transiently expressed in Arabidopsis mesophyll cell protoplasts. The protoplasts were incubated with or without 20 μM ABA for 15 min, and OST1/SnRK2.6 proteins were purified by immunoprecipitation using anti-GFP antibodies. After SDS-PAGE and CBB staining, OST1/SnRK2.6-GFP bands were excised and analyzed by LC-MS/MS¹⁷

### Analysis of Stomatal ABA Response

Infrared-based gas exchange analyzer systems were used including an integrated Multiphase Flash Fluorometer (Li-6800-01A or Li-6400; Li-Cor Inc.) for gas exchange analyses. Plants were grown on soil in Percival growth cabinets at a 12/12 h, 21° C./21° C. day/night cycle, a photosynthetic photon flux density of ˜90 mmol m-2 s-1, and 70 to 80% relative humidity for 6 to 7 weeks. Mature rosette leaves were detached at the basal part of petiole by a razor blade, and re-cut twice under distilled and deionized water. The petioles of the leaves were then immersed in ddH2O for gas exchange analysis. The detached leaves were clamped and the environment of the leaf chamber was controlled at 400 ppm ambient CO₂, 23-24° C., ˜65% relative air humidity, 150 μmol m⁻² s⁻¹ photon flux density, and 500 μmol s⁻¹ flow rate until stomatal conductance stabilized. One or 2 μM±-ABA was applied to the petiole for kinetic stomatal conductance response analyses as described⁵⁹.

### Patch-Clamp Analyses

Guard cell protoplasts from 4 to 6 week-old Arabidopsis plants were prepared^{24,33} ABA-activated S-type anion channel current recordings were carried out by using an Axon 200A amplifier (Axon instruments) and a Digidata 1440A low-noise data acquisition system. Epidermal tissues were isolated from one or two rosette leaves and collected using a nylon mesh (100-μm pore size). Subsequently the epidermal tissues were incubated in 10-ml protoplast isolation solution containing 500 mM D-mannitol, 1% cellulase R-10 (Yakult Pharmaceutical Industry), 0.5% macerozyme R-10 (Yakult Pharmaceutical Industry), 0.5% bovine serum albumin, 0.1% kanamycin sulfate, 0.1 mM CaCl₂), 0.1 mM KCl, and 10 mM ascorbic acid for 16 hr at 25° C. on a circular shaker at 50 rpm. Guard cell protoplasts were collected through a nylon mesh (10-μm pore size) and then washed two times with protoplast suspension solution containing 500 mM D-sorbitol, 0.1 mM CaCl₂), and 0.1 mM KCl (pH 5.6 with KOH) by centrifugation (200 g for 5 min at room temperature). Isolated guard cell protoplasts were stored on ice before use.

S-type anion currents in guard cell protoplasts were recorded using the whole-cell patch-clamp technique^{24,33}. The pipette solution was composed of 150 mM CsCl, 2 mM MgCl₂, 5.86 mM CaCl₂), 6.7 mM EGTA, and 10 mM Hepes-Tris (pH 7.1). 5 mM Mg-ATP was added to the pipette solution freshly before use. The bath solution was composed of 30 mM CsCl, 2 mM MgCl₂, 1 mM CaCl₂), and 10 mM MES-Tris (pH 5.6). Osmolalities of the pipette solution and the bath solution were adjusted to 500 mosmol kg⁻¹ and 485 mosmol kg⁻¹ using D-sorbitol, respectively. In FIG. 3, guard cell protoplasts were pre-incubated for 20 min in the bath solution containing 50 μM ABA prior to recordings, and ABA was added to the pipette solution. In Supplementary FIG. 10, guard cell protoplasts were pre-incubated for 30 min in the bath solution containing 10 μM ABA prior to recordings.

### Two-Electrode Voltage Clamp Recordings

The PCR amplified cDNA fragments of OST1, SLAC1, PYL9/RCAR1, ABI1, M3Kδ1, M3Kδ6 and M3Kδ7 were cloned into the oocyte expression vector pNB1 by using an advanced uracil-excision based cloning strategy as previously described⁶⁰. The mutant isoforms OST1-S171A, M3Kδ6-K775W and M3Kδ7-K740W were generated using the Quikchange Site-Directed Mutagenesis kit (Agilent Technologies). Linearized plasmids were used to generate cRNAs via the mMESSAGE mMACHINE® T7 kit (Thermo Fisher Scientific, Catalog number: AM1344). Surgically extracted ovaries of Xenopus laevis were ordered from Nasco (Fort Atkinson, Wis., product number: LM00935) and Ecocyte Bio Science US (Austin, Tex.) and oocytes were isolated as previously described⁶¹. 5 ng cRNA of each construct OST1, OST1-S171A, SLAC1, PYL9/RCAR1, ABI1 and 0.5 ng cRNAs of each construct M3Kδ1, M3Kδ6, M3Kδ7, M3Kδ6-K775W, M3Kδ7-K740W were co-injected into isolated oocytes in the indicated combinations. Oocytes were then incubated at 16° C. for 2 days in ND96 buffer (1 mM CaCl₂), 1 mM MgCl₂, 96 mM NaCl, 10 mM MES/Tris, pH=7.5). Osmolarity was adjusted to 220 mosmol kg⁻¹ by D-sorbitol. Using a Cornerstone (Dagan) TEV-200 amplifier and a Digidata 1440A low-noise data acquisition system with pClamp software (Molecular Devices), two-electrode voltage clamp recordings were performed in a bath solution containing 1 mM CaCl₂), 2 mM KCl, 24 mM NaCl, 70 mM Na-gluconate, 10 mM MES/Tris, pH 7.4, Osmolarity was adjusted to 220 mosmol kg⁻¹ by D-sorbitol. ABA was injected into oocytes to achieve a final concentration of 50 μM for analyses of ABA activation of SLAC1 currents. Steady state currents were recorded with 3 second voltage pulses ranging from +40 mV to −120 mV in −20 mV decrements, followed by a “tail” voltage of −120 mV and the holding potential was kept at 0 mV.

SLAC1-mediated currents in oocytes vary showing either time-dependent relaxation or more instantaneous currents when using a chloride bath solution^{61,62}. Furthermore, ion channel activities display different magnitudes from one oocyte batch to another due to protein expression level variation among batches of oocytes. To avoid time-of-measurement and inter-batch dependence in the data, H₂O-injected control and other indicated controls were included in each batch of oocytes and control experiments were recorded intermittently with the investigated conditions. Data from one representative oocyte batch are shown from the same batch in each figure panel and at least three independent batches of oocytes were investigated and showed consistent findings.

### Mesophyll Cell Protoplast Assays

Mesophyll cell protoplasts were isolated as described previously⁶³ from 3-4-week old Arabidopsis leaves. 10-20 μg of pUC18 plasmids carrying 35S:OST1/SnRK2.6-GFP:nosT or 35S:M3Kδ6-FLAG:nosT and 30 μg protoplasts were used for 20% PEG-mediated transient expression. After overnight incubation in incubation buffer (10 mM MES-KOH pH 6.0, 0.4 M mannitol, 20 mM KCl, 1 mM CaCl₂), protoplasts were incubated in 10 μM ABA or 0.8 M mannitol or in control buffer for 15 min and harvested by centrifugation at 13,000 g for 1 min. After the supernatants were removed, 20 μL SDS-PAGE loading buffer was added and incubated at 95° C. for 3 min.

### Measurements of Leaf Temperatures by Thermal Imaging

Plants grown 4-5 weeks on soil were sprayed with 20 μM ABA dissolved in water. After 3 hr under white light in the growth room, images were captured using an infrared thermal imaging camera (T650sc; FLIR, Wilsonville, Oreg.). Leaf temperatures were determined as average temperatures of each whole leaf area by using Fiji software (ImageJ version: 2.0.0-rc-59/1.51n).

### Creating CRISPR/Cas9-Based Knock-Out Arabidopsis

The m3kδ1 and m3kδ7 CRISPR/Cas9 deletion knock-out mutants were generated using CRISPR/Cas9 gene editing technology⁶⁴⁻⁶⁶ in the m3kδ6-2 mutant background. We used two guide RNAs to generate a large deletion in each target gene. The target sequences in M3Kδ1 were TACGGAAGCTCCACATCGGCGG (SEQ ID NO:3) and GATGCAAGTCGTTGGAGCTGTGG (SEQ ID NO:4) (PAM sites are underlined). Targets for M3Kδ7 were GACGGAGTTCCAGATCTCCGGG (SEQ ID NO:5) and CCAGAGAGCAGCAGTTCCCAGT (SEQ ID NO:6).

The designed m3kδ1crispr mutants were genotyped with the primer pair Delta1-GT1 and Delta1-GT2, which would generate a fragment of about 750 bp when the designed deletion took place. The primer pair could not amplify WT genomic DNA due to the large size of the fragment. To determine zygosity of m3kδ1crispr mutants, we used the primer set Delta1-GT1+Delta1-GT3, which amplifies a 777 bp fragment from WT DNA, but could not amplify a band in a homozygous mutant.

For m3kδ7crispr mutants, we used Delta7-GT1 and Delta7-GT4, which would generate a fragment of about 1390 bp if mutant DNA is used as PCR template. The primer pair could not amplify WT DNA because of the large fragment size. The Delta7-GT1 and Delta7-GT3 primer pair was able to generate a fragment of 1125 bp when WT DNA was used as PCR template. The Delta7-GT1/GT3 was used to differentiate homozygous m3kδ7crispr mutants from heterozygous m3kδ7crispr mutants. After isolating homozygous m3kδ1crispr m3kδ6-2 and m3kδ7crispr m3kδ6-2 mutants, these lines were crossed and homozygous triple mutants were recovered in the T2 generation. Primers for genotyping:

Delta1-GT1:
(SEQ ID NO: 7)
5′-TTGTTGGTTCCACGAACGGA-3′,
Delta1-GT2:
(SEQ ID NO: 8)
5′-GATGGCCGTAAATGCGGTTC-3′,
Delta1-GT3:
(SEQ ID NO: 9)
5′-CGGATCAGGATCAGAGACGC-3′,
Delta7-GT1:
(SEQ ID NO: 10)
5′-TGCATAAGGTGGTGAGCGAA-3′,
Delta7-GT3:
(SEQ ID NO: 11)
5′-CCAAACCCTGCATCCCAGAT-3′,
Delta7-GT4:
(SEQ ID NO: 12)
5′-GTCAAGGAAGAAGCGACCCA-3′.

Creating amiRNA Knock-Downs Targeting M3Kδ1, δ6 and δ7

The amiRNA sequence was designed using the WMD3 (http://wmd3.weigelworld.org/cgi-bin/webapp.cgi) and PHANTOM database (http://phantomdb.ucsd.edu). The amiRNA containing the target sequence (5′-TACGACTTGCATCGGGTTCAA-3′) (SEQ ID NO:13) for M3Kδ1, M3Kδ6 and M3Kδ7 was amplified by PCR using primers

(I:
(SEQ ID NO: 14)
5′-gaTACGACTTGCATCGGGTTCAAtactatttgtattcc-3′,
II:
(SEQ ID NO: 15)
5′-gaTTGAACCCGATGCAAGTCGTAtcaaagagaatcaatga-3′,
III:
(SEQ ID NO: 16)
5′-gaTTAAACCCGATGCTAGTCGTTtcacaggtcgtgatatg-3′,
IV:
(SEQ ID NO: 17)
5′-gaAACGACTAGCATCGGGTTTAAtctacatatatattcct-3′),

and inserted into the vector pFH0032²¹. Arabidopsis (Col-0) plants were used for floral-dip transformation. Three independent homozygous T3 seeds were used in the seed germination assays.

### BiFC Analyses

Constructs for BiFC analyses were generated by ligation of coding sequences of ABI1, RopGEF1, OST1/SnRK2.6, SnRK2.2, SnRK2.4, SnRK2.10, M3Kδ6, and M3Kδ7 into pSPYCE(M) or pSPYNE173 using the USER Cloning technology (see FIG. 23 for primer sequences). Plasmids were transformed into Agrobacterium tumefaciens (GV3101) and co-infiltrated with a plasmid expressing the silencing suppressor p19 in leaves of 6-week-old Nicotiana benthamiana plants. Subcellular localization analyses were performed using a Nikon Eclipse TE2000-U confocal microscope. Images were acquired using a Plan Apo VC 60XA/1.20 WI objective using identical settings (exposure time and gain). Three independent experiments were conducted where 3 leaves were analyzed for each combination. 5 z-stacks were acquired for each leaf. Maximum projections of z-stacks for each BiFC combination were quantified using Fiji and normalized over an infiltration control expressing p19 only.

### Statistics

Cotyledon greening assays were analyzed by two-way ANOVA followed by Tukey's tests. Leaf temperatures were analyzed by one-way ANOVA followed by Tukey's tests.

### FIGURE LEGENDS

FIG. 1A-G: Identification of M3Ks that reactivate OST1/SnRK2 kinases by phosphorylation.

a, Seeds of amiR-HsMYO wild-type (control line) or amiR-ax1117 mutant were sowed on ½ MS medium containing 2 μM ABA, or 0.02% EtOH as control, for germination assays. Representative images showing seed germination after 6 days. b, The percentage of seedlings showing green cotyledons was analyzed. Data represent mean±s.d. n=4 experiments. Each experiment included 64 seeds for each genotype. Letters at the top of columns are grouped based on two-way ANOVA and Tukey's test, P<0.05. c, Identification of the amiRNA sequence in amiR-ax1117 plants. Black box labels the sequence of amiR-ax1117. The amiR-ax1117 is predicted to include Raf-like protein kinase genes M3Kδ5, M3Kδ7, M3Kδ1, M3Kδ6 and M3KS-CTR1 kinase (see FIG. 7). d, Wild type (WT) and m3k amiRNA seedlings were incubated with 10 μM ABA for 15 min. In-gel kinase assays were performed using histone type III-S as a substrate. e, SnRK2 band intensities as shown in (d) were measured using ImageJ, n=3 experiments, error bars show +/−s.e.m. f, Recombinant GST-OST1/SnRK2.6 protein was dephosphorylated by alkaline phosphatase in vitro and used for in vitro phosphorylation assays after incubation with CPK6, CPK23 or MPK12 protein kinases. Note visible autophosphorylation activity of CPK6 and CPK23. g, Dephosphorylated recombinant His-OST1/SnRK2.6 protein was incubated with kinase domains of seven M3Ks and used for in-gel kinase assays (phylogenetic tree: see FIG. 7). Note lanes on the left are from the same gel as lanes in the middle section.

FIG. 2A-D: M3K-dependent OST1/SnRK2.6 Ser-171 phosphorylation is essential for ABA activation of OST1/SnRK2.6 activation.

a, The inactive M3Kδ6 kinase domain mutant (K775W) did not re-activate His-OST1/SnRK2.6 in vitro. b, Inactive GST-OST1/SnRK2.6-D140A kinase protein was incubated with M3Kδ6 kinase domain, and in vitro phosphorylation assays were performed with ³²P-ATP. c, Recombinant inactive OST1(D140A) and M3Kδ1 kinase domains were incubated with ATP. A mass spectrum of phosphorylated OST1 peptide (SSVLHpSQPK) is shown. pS indicates phosphorylated Ser171 of OST1(D140A). d, Phosphorylation at Ser171 was not detectable after in vitro auto-phosphorylation of OST1/SnRK2.6, but was consistently phosphorylated in the presence of M3Kδ1. e, OST1(S171A)-GFP was transiently expressed in Arabidopsis mesophyll cell protoplasts. Protoplasts were incubated with 10 μM ABA or control buffer for 15 min, and OST1/SnRK2.6 activities were analyzed by in-gel kinase assays.

FIG. 3A-F: a, Stomatal conductances were analyzed in intact detached leaves of stable transgenic Arabidopsis [pUBQ10:OST1-HF ost1-3 (OST1-comp2) and pUBQ10:OST1-S171A-HF ost1-3 (S171A-comp2)]. 2 μM ABA was applied to petioles at 0 min. Data presented are means±s.e.m. (n=4 leaves from 4 plants for each genotype). b, Leaf temperatures of Col, ost1-3, OST1-comp2 and S171A-comp2 were measured by thermal imaging. Plants were sprayed with 20 μM ABA, and thermal images were taken after 3 hr. The bright field image shows where leaves from neighboring plants over-lapped. c, Leaf temperatures were measured by using Fiji software (n=5 experiments, means+/−s.e.m.). Letters at the top of columns are grouped based on one-way ANOVA and Tukey's test, P<0.05. d, ABA-activated S-type anion channel currents were investigated by patch-clamp analyses using guard cell protoplasts from the transgenic Arabidopsis lines pUBQ10:OST1-HF ost1-3 (OST1-WT) and pUBQ0:OST1-S171A-HF ost1-3 (OST1-S171A). e, Average current voltage relationship of S-type anion channel as shown (d). Data presented are means+/−s.e.m. f, Kinase activities of OST1(S171A) in mesophyll cells from stably-transformed homozygous transgenic plants were investigated by in-gel kinase assays. Protoplasts were incubated with 10 μM ABA for 15 min.

FIG. 4A-F: MAPKK-kinases are essential for ABA signalling module.

a and b, In vitro reconstitution of ABA-induced OST1/SnRK2 activation without M3Kδ6 (a) or with M3Kδ6 (b). The recombinant proteins His-PYR1/RCAR11, His-OST1/SnRK2.6 without (a) or with (b) GST-M3Kδ6 kinase domain were mixed. After addition of His-HAB1, protein solutions were incubated for 10 min. Then, 50 μM ABA was added to the protein solution. Reactions were stopped at the indicated times. OST1/SnRK2.6 kinase activities were detected by in-gel kinase assays. c, Recombinant His-PYR1/RCAR11, His-HAB1, His-OST1/SnRK2.6, His-AKS1 and GST-M3Kδ6 kinase domain were mixed as indicated above the gel. 50 μM ABA was added before (lane 5) or after (lanes 6 and 8) 10 min incubation at room temperature. Then, 100 μM ATP was added (lanes 2 to 8) to trigger phosphorylation reactions for 10 min. Note that M3Kδ6 is required for ABA-induced AKS1 phosphorylation when ABA is added 10 min after exposure to HAB1-PP2C-including mix (compare lanes 6 and 8). Reactions were stopped by addition of SDS-PAGE loading buffer. Phosphorylation of AKS1 is detected by binding of 14-3-3Phi (At1g35160) to the phosphorylated AKS1 protein¹⁵. AKS1 phosphorylation is shown by protein-blot (top), and protein amount is monitored by immuno-blot (bottom). d-f, Reconstitution of ABA-activation of SLAC1 channels in Xenopus oocytes, in the presence or absence of M3Ks. (d) Representative whole cell chloride current recordings of oocytes co-expressing the indicated proteins, without (control) or with injection of 50 μM ABA (+ABA). Currents were recorded in response to voltage pulses ranging from +40 mV to −120 mV in −20 steps with a holding potential at 0 mV and a final tail potential of −120 mV. (e) Mean current-voltage curves of oocytes co-expressing the indicated proteins, with or without injection of ABA. The symbols of H₂O control, OST1+SLAC1, PYL9/RCAR1+ABI1+OST1+SLAC1, PYL9/RCAR1+ABI1+OST1+SLAC1+ABA and PYL9/RCAR1+ABI1+OST1+SLAC1+M3Ks overlapped. Single symbols are shown for some data points for better viewing. (f) Average SLAC1-mediated currents at −100 mV, co-expressing the indicated proteins, in the presence or absence of 50 μM ABA.

Data from 3 independent batches of oocytes showed similar results. One representative batch of oocytes is shown, with the number of oocytes in that batch indicated in parentheses. H₂O, OST1+SLAC1, PYL9/RCAR1+ABI1+OST1+SLAC1 and PYL9/RCAR1+ABI1+OST1+SLAC1+ABA controls are the same data in both panels in (e) as the data are from the same oocyte batch. Error bars denote mean±s.e.m. Means with letters (a, b, c and d) are grouped based on one-way ANOVA and Tukey's multiple comparisons test, P<0.05.

FIG. 5A-I: MAPKK-kinases are required for plant ABA response.

a, Genome structures and T-DNA insertion sites of M3K genes are shown. b, Genomic regions of CRISPR/Cas9-mediated M3Kδ1 and M3Kδ7 gene deletions are shown. These deletions were introduced in the m3kδ6-2 T-DNA knock out mutant as a background. c, RT-PCR assays show transcripts of kinase domains of M3Ks in the m3kδ1crispr m3kδ6-2 m3kδ7crispr triple mutant. d, m3kδ1crispr m3kδ6-2 m3kδ7crispr triple mutant seedlings were grown on ½ MS plates supplemented with 2 μM ABA or ethanol (control) for 16 days. e, Seedlings showing green cotyledons as in (d) were counted. n=3 (EtOH) and n=4 (ABA) experiments, means+/−s.d., 45 seeds per genotype were used in each experiment. f, RT-PCR shows M3Kδ1, δ6 and δ7 expression in the indicated m3k T-DNA insertion mutants. δ6(KD) refers to primers that amplify the M3Kδ6 kinase domain in the m3kδ6-1 T-DNA line. g, m3kδ1 m3kδ6-1 m3kδ7 T-DNA triple mutant plants were grown on ½MS plates supplemented with 0.8 μM ABA for 9 days. h, Seedlings showing green cotyledons as in (g) were counted. n=3, means+/−s.d., 60-88 seeds were used per genotype in each assay. i, Three amiRNA lines targeting M3Kδ1, 66 and 67 were grown on ½MS plates supplemented with EtOH (control) or 2 μM ABA for 9 days. As a control line, the amiRNA-HsMYO line²¹ was used. j, Seedlings showing green cotyledons as in (i) were counted. n=3 (EtOH) and 4 (ABA) experiments, means+/−s.d., 81 seeds per genotype were analyzed in each experiment. (e, h and i) Letters at the top of columns are grouped based on two-way ANOVA and Tukey's test, P<0.05.

FIG. 6A-F: MAPKK-kinases mediate ABA- and osmotic stress-induced SnRK2 activation in planta.

a, m3kδ1crispr m3kδ6-2 m3kδ7crispr triple mutant seedlings were incubated in 10 μM ABA or 0.3 M mannitol (Osmo) for 15 min. SnRK2 activities were tested by in-gel kinase assays. Arrowhead shows SnRK2 activity²³. b, Normalized band intensities as shown in (a) were measured by using ImageJ. n=4, means+/−s.e.m. c, m3kδ1 m3kδ6-1 m3kδ7 T-DNA triple mutant seedlings were incubated in 10 μM ABA or 0.3 M mannitol (Osmo) for 15 min. SnRK2 activities were analyzed by in-gel kinase assays. d, Normalized band intensities as shown in (c) were measured by using ImageJ. n=4 experiments, means+/−s.e.m. e, Recombinant GST-tagged Arabidopsis SnRK2 protein kinases were incubated with M3Kδ1 kinase domain. SnRK2 kinase activities were analyzed by in-gel kinase assays. f, M3Kδ6-FLAG was transiently expressed in Arabidopsis mesophyll cell protoplasts. Protoplasts were incubated in 0.8 M mannitol (Osmo) for 15 min. M3Kδ6 proteins were detected by immuno-blot using anti-FLAG antibody. In the Osmo lane, the M3Kδ6 band showed a slight mobility shift as indicated by an asterisk.

FIG. 7: Phylogenetic tree of Arabidopsis Raf-like MAPKK kinases.

All M3Ks in subgroup B and selected M3Ks in subgroup C1-7 are shown (see e.g., MAPK Group, Mitogen-activated protein kinase cascades in plants: a new nomenclature. Trends Plant Sci 7, 301-308 (2002)) # and * indicates genes targeted by the amiR-ax1117, and the m3k amiRNA, respectively.

FIG. 8: Biological replicate example of SnRK2 activities in m3k amiRNA line. Replicate of in-gel kinase assay using m3k amiRNA line and WT (Col-0 accession) is shown. See FIG. 1d for an independent experiment.

FIG. 9: Full-length M3Kδ1 activates OST1/SnRK2.6 in vitro. Recombinant GST-OST1 and full-length His-M3Kδ1 (M3Kδ1_Full) or GST-M3Kδ1_KD (kinase domain) were incubated in the presence of ATP for 30 min. OST1/SnRK2.6 activity was measured by in-gel kinase assays. Note that the truncated kinase domain (M3Kδ1_KD) has a higher activity than the full length M3Kδ1 protein (M3Kδ1_Full). CBB shows loading control. The band labeled by an asterisk showing a similar mobility to the GST-M3Kδ1_KD in the right lane may be a degradation product of His-M3Kδ1.

FIG. 10: M3Kδ1, 66 and 67 directly phosphorylate OST1/SnRK2.6.

Recombinant kinase inactive GST-OST1/SnRK2.6 (D140A) protein was incubated with M3Kδ1, M3Kδ6 or M3Kδ7 kinase domains, and in vitro phosphorylation assays were performed with ³²P-ATP.

FIG. 11A-C: Ser-171 is important for ABA-activation of OST1/SnRK2.6 but not for in vitro protein kinase enzyme activity.

a, Recombinant GST-OST1/SnRK2.6 proteins carrying S171A, S175A or T176A mutation were used for in vitro autophosphorylation. OST1/SnRK2.6 (S175A) has no kinase activity (Belin, C. et al. Plant Physiol 141, 1316-1327 (2006)). b, OST1/SnRK2.6-GFP variants (WT, S171A, S175A or T176A) were transiently expressed in Arabidopsis mesophyll cell protoplasts. Protoplasts were incubated in 10 μM ABA for 15 min. OST1/SnRK2.6 protein kinase activity was detected by in-gel kinase assays (top panel). OST1/SnRK2.6-GFP proteins were detected by immuno-blot using GFP antibody (bottom panel). c, OST1-GFP (S171E) kinase activity was tested as shown in (b).

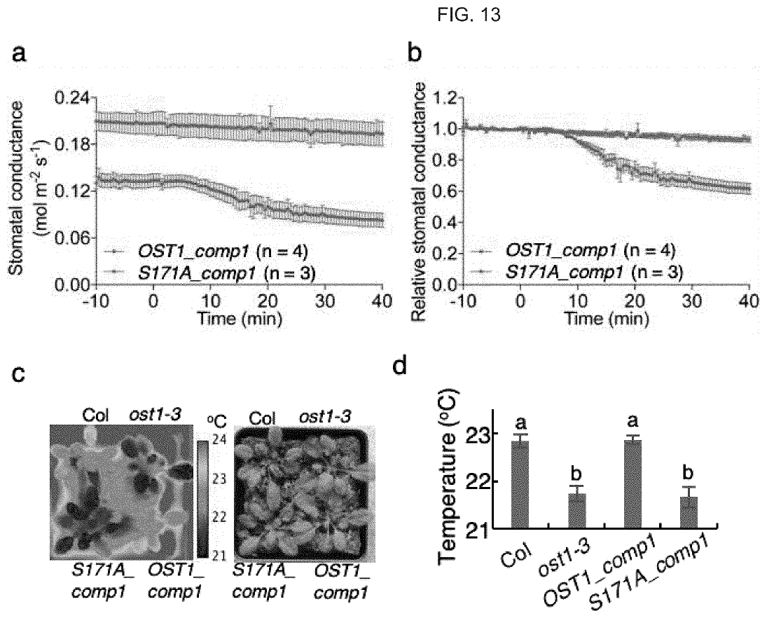

FIG. 12A-B: ABA induces phosphorylation at Ser-171 in OST1/SnRK2.6.

a, The sequence of identified phosphorylated peptide by mass spectrometry. OST1/SnRK2.6-GFP was expressed in Arabidopsis mesophyll cell protoplasts and purified by immunoprecipitation with GFP antibodies before or after 20 μM ABA treatment for 15 min. S(+79.97) indicates phosphorylated serine residues. Values (Control and ABA) indicate normalized peak areas for phosphorylated peptides. The phosphorylated peptide was not detected in the control sample. In contrast the peptide was clearly phosphorylated in response to ABA. b, An annotated mass spectrum of the phosphorylated peptide in the presence of ABA is shown.

FIG. 13A-D: Stomatal conductances and leaf temperatures of independent transgenic ost1-3 Arabidopsis lines expressing OST1(WT or S171A)-HF.

a, Stomatal conductances were analyzed in detached leaves of stable transgenic Arabidopsis [pUBQ10:OST1-HF ost1-3 (OST1_comp1) or pUBQ10:OST1(S171A)-HF ost1-3 (S171A_comp1)]. 2 μM ABA was applied at 0 min. Different independent transgenic lines from those shown in FIG. 3a were used. b, Relative stomatal conductances in (a) normalized to the average of the 10 minutes before addition of ABA. Data presented are mean±s.e.m. from n=3 to 4 leaves from independent plants in each genotype. c, Leaf temperatures of homozygous transgenic Arabidopsis lines (OST1_comp1 and S171A_comp1) were measured by thermal imaging. Plants were sprayed with 20 μM ABA, and thermal images were taken after 3 hr. The bright field image shows where leaves from neighboring plants over-lapped. d, Leaf temperatures were measured by using Fiji software (n=5 experiments, means+/−s.e.m.). Letters at the top of columns are grouped based on one-way ANOVA and Tukey's test, P<0.05.

FIG. 14A-G: M3Kδs activate SLAC1 channels together with OST1/SnRK2.6 in Xenopus oocytes.

a, Representative whole cell chloride current recordings of oocytes injected with the indicated cRNAs. Currents were recorded in response to voltage pulses ranging from +40 mV to −120 mV in −20 mV steps with a holding potential at 0 mV and a final tail potential of −120 mV. b-d, Mean current-voltage curves of oocytes co-expressing OST1 and SLAC1, in the presence or absence of the indicated M3K proteins. In panel (b) and (d), the symbols of H₂O, OST1+SLAC1 and SLAC1+M3Ks overlapped. One symbol is shown for some data points for better viewing. (e) Average SLAC1 mediated currents at −100 mV, co-expressing OST1, in the presence or absence of the indicated M3K proteins. M3K6 and OST1 and/or SLAC1 cRNA were injected at a concentration ratio of 1 to 10 to 10 (see main text). Data from one representative batch of oocytes are shown, with the number of oocytes in that batch indicated in parentheses. Control H₂O and OST1+SLAC1 data are the same data in (b), (c) and (d), as these data are from the same batch of oocytes (see Methods). Four independent batches of oocytes showed similar results. Error bars denote mean±s.e.m. f, Mean current-voltage curves of Xenopus oocytes injected with the indicated cRNAs. g, Average SLAC1-mediated currents from panel (f) at −120 mV, co-expressing OST1 isoforms, in the presence of M3Kδ1. The injected cRNA ratio of M3Kδ1 and OST1 isoforms was 1 to 10 (see main text). Letters on the bottom of columns are grouped based on one-way ANOVA with Tukey's test, P<0.01.

FIG. 15A-E: M3K-dependent S-type anion channel activation is dependent on the M3K kinase activities and the Ser171 residue in the OST1/SnRK2.6 activation loop.

a-d, Mean current-voltage curves of Xenopus oocytes injected with the indicated cRNAs. The symbols of OST1-S171A+SLAC1+M3Kδ6 are not visible because the symbols overlap. e, Average SLAC1-mediated currents from panels (a) to (d) at −100 mV, co-expressing OST1 or the kinase inactive OST1-S171A mutant isoform, in the presence of the indicated M3Ks or the kinase-inactive M3Kδ6-K775W and M3Kδ7-K740W isoforms. When M3K6 and OST1 cRNAs were co-injected the ratio of M3K6 and OST1 cRNA was 1 to 10 (see main text). Data from one representative oocyte batch are shown. Results from 3 independent batches of oocytes showed similar results. H₂O, SLAC1 and OST1+SLAC1 controls are the same in panels (a), (b), (c) and (d), as these were recorded in the same batch of oocytes. Error bars denote mean±s.e.m. FIG. 14E graphically illustrates the results of the data from FIG. 14A-D.

FIG. 16A-H: ABA-induced stomatal closing and activation of guard cell S-type anion channels are impaired m3k amiRNA line.

a, Leaves from m3k amiRNAi and the control amiRNA-HsMYO line (expressing an amiRNA targeting human myosin 2), which has no target gene in Arabidopsis plants were analyzed in time-resolved stomatal conductance analyses in which 1 μM ABA was added to the transpiration stream via the petiole (as described in e.g., Ceciliato, P. et al. Plant Methods 15, doi:10.1186/s13007-019-0423-y (2019)) as indicted by the red arrowhead (n=3 leaves from 3 independent plants per genotype, +/−s.d.). b, Normalized relative stomatal conductance to the first data point shown in (a). c-h, ABA-activated S-type anion channel currents were investigated by patch-clamp analyses using guard cell protoplasts from the wildtype parent Col-0 (c, d), the HsMYO amiRNA control line (e, f), and the m3k amiRNA line (g, h). Representative current traces (c, e, g) and average current voltage relationships (d, f, h) of S-type anion channel currents are shown. Data presented are means+/−s.e.m.

FIG. 17A-C: m3k double mutants show weak ABA-insensitive phenotypes.

a, m3k double (m3kδ6-2/δ7) and triple (m3kδ1/δ6-1/δ7) mutants were grown on ½ MS plates supplemented with 2 μM ABA or EtOH for 9 days. Seedlings showing green cotyledons were counted. n=3 experiments, means+/−s.d., 45-48 seeds were used per genotype and condition in each experiment. b, m3k double (m3kδ1/δ7) and triple (m3kδ1 crispr δ6-2/δ7crispr) mutants were grown on ½ MS plates supplemented with 2 μM ABA or EtOH for 16 days. Seedlings showing green cotyledons were counted. n=3 experiments (EtOH) and n=4 experiments (ABA), means+/−s.d., 45 seeds were used per genotype and condition in each experiment. The crispr and Col results are the same as FIG. 5e because they were grown on the same plates. Letters at the top of columns are grouped based on two-way ANOVA and Tukey's test, P<0.05. c, Wild type and m3k9J δ6-1/δ7 triple mutant seedlings were grown on ½ MS plates for three days and transferred to ½ MS plates with or without 20 μM ABA followed by an additional seven-day incubation. Primary root length was measured using ImageJ software. n=5 experiments, means+/−s.e.m., 10-13 seedlings per genotype were used in each experiment. Letters at the top of columns are grouped based on two-way ANOVA and Tukey's test, P<0.05.

FIG. 18: Biological replicate example of SnRK2 activities in m3k9J δ6-1/δ7 triple mutant line.

Replicate of in-gel kinase assay using m3k9J δ6-1/δ7 triple mutant is shown. See FIG. 6c for an independent experiment. Arrowhead indicates SnRK2 kinase activities.

FIG. 19A-B: m3k mutants show reduced sensitivity to osmotic stress in seed germination.

a, m3kδ1/δ7 double mutant and m3kδ1crispr δ6-2/δ7crispr triple mutant seedlings were grown on ½ MS plates supplemented with 0.4 M mannitol for 3 days. Green cotyledons were counted. n=4 experiments, means+/−s.d., 64 seeds per genotype were used in each experiment. b, Three amiRNA lines targeting M3Kδ1, 66 and 67 were grown on ½ MS plate supplemented with 0.4 M mannitol for 3 days. As a control line, HsMYO was used. n=4 experiments, means+/−s.d., 64 seeds per genotype were used in each experiment. Letters at the top of columns are grouped based on two-way ANOVA and Tukey's test, P<0.05.

FIG. 20A-B: M3Ks activate SnRK2.3 in vitro.

a, GST-SnRK2.3 protein was incubated with the kinase domains of M3Kδ1, M3Kδ6 or M3Kδ7 and in-gel kinase assays were conducted. b, SnRK2.2-GFP (WT or S180A) and SnRK2.3-GFP (WT or S172A) were expressed in Arabidopsis mesophyll cell protoplasts and purified by immunoprecipitation with GFP antibodies. The isolated proteins bound on magnetic immunoprecipitation beads were used for in vitro phosphorylation assays using histone as an artificial substrate. Phosphorylation reactions were started by the addition of ³²P-ATP. After 30 min, reactions were stopped by the addition of 3×SDS-PAGE sample buffer.

FIG. 21A-D: M3Ks interact with SnRK2 kinases in BiFC experiments in plant cells.

a, Co-immunoprecipitation experiments using transiently expressed M3Kδ6 and OST1/SnRK2.6 in Arabidopsis mesophyll cell protoplasts. OST1/SnRK2.6-GFP or GFP control co-expressed with M3Kδ6-FLAG were immunoprecipitated with GFP antibodies. Precipitated proteins were analyzed by immunoblots using GFP or FLAG antibody. b and c, BiFC analyses of nYFP-M3Kδ6 (b) or nYFP-M3Kδ7 (c) with OST1/SnRK2.6-cYFP, SnRK2.2-cYFP, 2.4-cYFP and 2.10-cYFP infiltrated in 6-week-old Nicotiana benthamiana leaves. nYFP-GEF1/cYFP-ABI1 combination was used as a positive control. All images are at the same scale. Scale bars=50 μm. d and e, BiFC quantifications measured from maximal projections of z-stacks and normalized over an infiltration control expressing p19 only. BiFC quantifications were analyzed by one-way ANOVA followed by Tukey's tests. Confocal images were acquired using identical settings for each BiFC experiment. Means±s.e.m. (n=45).

FIG. 22: A-B: m3k quadruple mutant shows an ABA-insensitive phenotype in cotyledon emergence.

a, m3k triple (m3kδ1/δ6-1/δ7) and m3k quadruple (m3kδ1/δ5/δ6-1/δ7) mutant plants were grown on ½ MS plates supplemented with 0.8 μM ABA for 6 days (m3kδ5=SALK_025685). Green emerging cotyledons were counted. n=6 experiments, means+/−s.d., 81 seeds were used per genotype and per condition in each experiment.

Letters at the top of columns are grouped based on two-way ANOVA and Tukey's test, P<0.05. b, Gene expression levels of B3 subgroup M3K genes and three SnRK2 genes in guard cells and mesophyll cells. Data were obtained from the public microarray database eFP Browser (http://bar.utoronto.ca/efp/cgi-bin/efpWeb.cgi), see Yang et al. Plant Methods 4, 6, doi:10.1186/1746-4811-4-6 (2008).

### REFERENCES

1 Finkelstein, R. Abscisic Acid synthesis and response. Arabidopsis Book 11, e0166, doi:10.1199/tab.0166 (2013).
2 Cutler, S. R., Rodriguez, P. L., Finkelstein, R. R. & Abrams, S. R. Abscisic acid: emergence of a core signaling network. Annu Rev Plant Biol 61, 651-679, doi:10.1146/annurev-arplant-042809-112122 (2010).
3 Raghavendra, A. S., Gonugunta, V. K., Christmann, A. & Grill, E. ABA perception and signalling. Trends Plant Sci 15, 395-401, doi:10.1016/j.tplants.2010.04.006 (2010).
4 Ma, Y. et al. Regulators of PP2C phosphatase activity function as abscisic acid sensors. Science 324, 1064-1068 (2009).
5 Park, S. Y. et al. Abscisic acid inhibits type 2C protein phosphatases via the PYR/PYL family of START proteins. Science 324, 1068-1071 (2009).
6 Weiner, J. J., Peterson, F. C., Volkman, B. F. & Cutler, S. R. Structural and functional insights into core ABA signaling. Curr Opin Plant Biol 13, 495-502, doi:10.1016/j.pbi.2010.09.007 (2010).
7 Joshi-Saha, A., Valon, C. & Leung, J. Abscisic acid signal off the STARting block. Mol Plant 4, 562-580, doi:10.1093/mp/ssr055 (2011).
8 Tischer, S. V. et al. Combinatorial interaction network of abscisic acid receptors and coreceptors from Arabidopsis thaliana. Proc Natl Acad Sci USA 114, 10280-10285, doi:10.1073/pnas.1706593114 (2017).
9 Vlad, F. et al. Protein phosphatases 2C regulate the activation of the Snf1-related kinase OST1 by abscisic acid in Arabidopsis. Plant Cell 21, 3170-3184, doi:10.1105/tpc.109.069179 (2009).
10 Umezawa, T. et al. Type 2C protein phosphatases directly regulate abscisic acid-activated protein kinases in Arabidopsis. Proc Natl Acad Sci USA 106, 17588-17593, doi:10.1073/pnas.0907095106 (2009).
11 Vlad, F. et al. Phospho-site mapping, genetic and in planta activation studies reveal key aspects of the different phosphorylation mechanisms involved in activation of SnRK2s. Plant J 63, 778-790, doi:10.1111/j.1365-313X.2010.04281.x (2010).
12 Geiger, D. et al. Activity of guard cell anion channel SLAC1 is controlled by drought-stress signaling kinase-phosphatase pair. Proc Natl Acad Sci USA 106, 21425-21430, doi:10.1073/pnas.0912021106 (2009).
13 Lee, S. C., Lan, W., Buchanan, B. B. & Luan, S. A protein kinase-phosphatase pair interacts with an ion channel to regulate ABA signaling in plant guard cells. Proc Natl Acad Sci USA 106, 21419-21424, doi:10.1073/pnas.0910601106 (2009).
14 Fujii, H. et al. In vitro reconstitution of an abscisic acid signalling pathway. Nature 462, 660-664, doi:10.1038/nature08599 (2009).
15 Takahashi, Y. et al. bHLH transcription factors that facilitate K⁺ uptake during stomatal opening are repressed by abscisic acid through phosphorylation. Sci Signal 6, ra48, doi:10.1126/scisignal.2003760 (2013).
16 Belin, C. et al. Identification of features regulating OST1 kinase activity and OST1 function in guard cells. Plant Physiol 141, 1316-1327, doi:10.1104/pp. 106.079327 (2006).
17 Brandt, B. et al. Reconstitution of abscisic acid activation of SLAC1 anion channel by CPK6 and OST1 kinases and branched ABI1 PP2C phosphatase action. Proc Natl Acad Sci USA 109, 10593-10598, doi:10.1073/pnas.1116590109 (2012).
18 Takahashi, Y., Ebisu, Y. & Shimazaki, K. I. Reconstitution of Abscisic Acid Signaling from the Receptor to DNA via bHLH Transcription Factors. Plant Physiol 174, 815-822, doi:10.1104/pp. 16.01825 (2017).
19 Boudsocq, M., Barbier-Brygoo, H. & Lauriere, C. Identification of nine sucrose nonfermenting 1-related protein kinases 2 activated by hyperosmotic and saline stresses in Arabidopsis thaliana. J Biol Chem 279, 41758-41766, doi:10.1074/jbc.M405259200 (2004).
20 Yoshida, R. et al. The regulatory domain of SRK2E/OST1/SnRK2.6 interacts with ABI1 and integrates abscisic acid (ABA) and osmotic stress signals controlling stomatal closure in Arabidopsis. J Biol Chem 281, 5310-5318, doi:10.1074/jbc.M509820200 (2006).
21 Hauser, F. et al. A Genomic-Scale Artificial MicroRNA Library as a Tool to Investigate the Functionally Redundant Gene Space in Arabidopsis. Plant Cell 25, 2848-2863, doi:tpc.113.112805 [pii] 10.1105/tpc.113.112805 (2013).
22 MAPK Group. Mitogen-activated protein kinase cascades in plants: a new nomenclature. Trends Plant Sci 7, 301-308 (2002).
23 Fujii, H. & Zhu, J. K. Arabidopsis mutant deficient in 3 abscisic acid-activated protein kinases reveals critical roles in growth, reproduction, and stress. Proc Natl Acad Sci USA 106, 8380-8385, doi:10.1073/pnas.0903144106 (2009).
24 Brandt, B. et al. Calcium specificity signaling mechanisms in abscisic acid signal transduction in Arabidopsis guard cells. Elife 4, doi:10.7554/eLife.03599 (2015).
25 Geiger, D. et al. Guard cell anion channel SLAC1 is regulated by CDPK protein kinases with distinct Ca2+ affinities. Proc Natl Acad Sci USA 107, 8023-8028, doi:10.1073/pnas.0912030107 (2010).
26 Jammes, F. et al. MAP kinases MPK9 and MPK12 are preferentially expressed in guard cells and positively regulate ROS-mediated ABA signaling. Proc Natl Acad Sci USA 106, 20520-20525, doi:10.1073/pnas.0907205106 (2009).
27 Des Marais, D. L. et al. Variation in MPK12 affects water use efficiency in Arabidopsis and reveals a pleiotropic link between guard cell size and ABA response. Proc Natl Acad Sci USA 111, 2836-2841, doi:10.1073/pnas.1321429111 (2014).
28 Saruhashi, M. et al. Plant Raf-like kinase integrates abscisic acid and hyperosmotic stress signaling upstream of SNF1-related protein kinase2. Proc Natl Acad Sci USA 112, E6388-6396, doi:10.1073/pnas.1511238112 (2015).
29 Yoshida, R. et al. ABA-activated SnRK2 protein kinase is required for dehydration stress signaling in Arabidopsis. Plant Cell Physiol 43, 1473-1483 (2002).
30 Mustilli, A. C., Merlot, S., Vavasseur, A., Fenzi, F. & Giraudat, J. Arabidopsis OST1 protein kinase mediates the regulation of stomatal aperture by abscisic acid and acts upstream of reactive oxygen species production. Plant Cell 14, 3089-3099 (2002).
31 Laanemets, K. et al. Calcium-dependent and -independent stomatal signaling network and compensatory feedback control of stomatal opening via Ca2+ sensitivity priming. Plant Physiol 163, 504-513, doi:10.1104/pp. 113.220343 (2013).
32 Laanemets, K. et al. Mutations in the SLAC1 anion channel slow stomatal opening and severely reduce K+ uptake channel activity via enhanced cytosolic [Ca2+] and increased Ca2+ sensitivity of K+ uptake channels. New Phytol 197, 88-98, doi:10.1111/nph.12008 (2013).
33 Pei, Z. M., Kuchitsu, K., Ward, J. M., Schwarz, M. & Schroeder, J. I. Differential abscisic acid regulation of guard cell slow anion channels in Arabidopsis wild-type and abi1 and abi2 mutants. Plant Cell 9, 409-423, doi:10.1105/tpc.9.3.409 (1997).
34 Fujii, H., Verslues, P. E. & Zhu, J. K. Arabidopsis decuple mutant reveals the importance of SnRK2 kinases in osmotic stress responses in vivo. Proc Natl Acad Sci USA 108, 1717-1722, doi:10.1073/pnas.1018367108 (2011).
35 de Oliveira, P. S. et al. Revisiting protein kinase-substrate interactions: Toward therapeutic development. Sci Signal 9, re3, doi:10.1126/scisignal.aad4016 (2016).
36 Danquah, A. et al. Identification and characterization of an ABA-activated MAP kinase cascade in Arabidopsis thaliana. Plant J 82, 232-244, doi:10.1111/tpj.12808 (2015).
37 Matsuoka, D., Yasufuku, T., Furuya, T. & Nanmori, T. An abscisic acid inducible Arabidopsis MAPKKK, MAPKKK18 regulates leaf senescence via its kinase activity. Plant Molecular Biology 87, 565-575, doi:10.1007/s11103-015-0295-0 (2015).
38 Li, K. et al. AIK1, A Mitogen-Activated Protein Kinase, Modulates Abscisic Acid Responses through the MKK5-MPK6 Kinase Cascade. Plant Physiology 173, 1391-1408, doi:10.1104/pp. 16.01386 (2017).
39 Shitamichi, N., Matsuoka, D., Sasayama, D., Furuya, T. & Nanmori, T. Over-expression of MAP3K delta 4, an ABA-inducible Raf-like MAP3K that confers salt tolerance in Arabidopsis. Plant Biotechnology 30, 111-118, doi:10.5511/plantbiotechnology.13.0108a (2013).
40 Lee, S., Lee, M., Kim, J. & Kim, S. Arabidopsis Putative MAP Kinase Kinase Kinases Raf10 and Raf11 are Positive Regulators of Seed Dormancy and ABA Response. Plant and Cell Physiology 56, 84-97, doi:10.1093/pcp/pcu148 (2015).
41 Amagai, A. et al. Phosphoproteomic profiling reveals ABA-responsive phosphosignaling pathways in Physcomitrella patens. Plant J 94, 699-708, doi:10.1111/tpj.13891 (2018).
42 Shinozawa, A. et al. SnRK2 protein kinases represent an ancient system in plants for adaptation to a terrestrial environment. Commun Biol 2, 30, doi:10.1038/s42003-019-0281-1 (2019).
43 Boudsocq, M., Droillard, M. J., Barbier-Brygoo, H. & Lauriere, C. Different phosphorylation mechanisms are involved in the activation of sucrose nonfermenting 1 related protein kinases 2 by osmotic stresses and abscisic acid. Plant Mol Biol 63, 491-503, doi:10.1007/s11103-006-9103-1 (2007).
44 Yamaguchi-Shinozaki, K. & Shinozaki, K. Transcriptional regulatory networks in cellular responses and tolerance to dehydration and cold stresses. Annu Rev Plant Biol 57, 781-803, doi:10.1146/annurev.arplant.57.032905.105444 (2006).
45 Liu, Q. et al. Two transcription factors, DREB1 and DREB2, with an EREBP/AP2 DNA binding domain separate two cellular signal transduction pathways in drought- and low-temperature-responsive gene expression, respectively, in Arabidopsis. Plant Cell 10, 1391-1406 (1998).
46 Krzywinska, E. et al. Phosphatase ABI1 and okadaic acid-sensitive phosphoprotein phosphatases inhibit salt stress-activated SnRK2.4 kinase. BMC Plant Biol 16, 136, doi:10.1186/s12870-016-0817-1 (2016).
47 Krzywinska, E. et al. Protein phosphatase type 2C PP2CA together with ABI1 inhibits SnRK2.4 activity and regulates plant responses to salinity. Plant Signal Behav 11, e1253647, doi:10.1080/15592324.2016.1253647 (2016).
48 Ruschhaupt, M. et al. Rebuilding core abscisic acid signaling pathways of Arabidopsis in yeast. EMBO J 38, e101859, doi:10.15252/embj.2019101859 (2019).
49 Waadt, R. et al. FRET-based reporters for the direct visualization of abscisic acid concentration changes and distribution in Arabidopsis. eLife 3, e01739, doi:10.7554/eLife.01739 (2014).
50 Zhao, Y. et al. Arabidopsis Duodecuple Mutant of PYL ABA Receptors Reveals PYL Repression of ABA-Independent SnRK2 Activity. Cell Rep 23, 3340-3351.e3345, doi:10.1016/j.celrep.2018.05.044 (2018).
51 Yuan, F. et al. OSCA1 mediates osmotic-stress-evoked Ca2+ increases vital for osmosensing in Arabidopsis. Nature 514, 367-371, doi:10.1038/nature13593 (2014).
52 Choi, W. G., Toyota, M., Kim, S. H., Hilleary, R. & Gilroy, S. Salt stress-induced Ca2+ waves are associated with rapid, long-distance root-to-shoot signaling in plants. Proc Natl Acad Sci USA 111, 6497-6502, doi:10.1073/pnas.1319955111 (2014).
53 Stephan, A. B., Kunz, H. H., Yang, E. & Schroeder, J. I. Rapid hyperosmotic-induced Ca2+ responses in Arabidopsis thaliana exhibit sensory potentiation and involvement of plastidial KEA transporters. Proc Natl Acad Sci USA 113, E5242-5249, doi:10.1073/pnas.1519555113 (2016).
54 Kudla, J. et al. Advances and current challenges in calcium signaling. New Phytol 218, 414-431, doi:10.1111/nph.14966 (2018).
55 Yoshida, T., Mogami, J. & Yamaguchi-Shinozaki, K. ABA-dependent and ABA-independent signaling in response to osmotic stress in plants. Curr Opin Plant Biol 21, 133-139, doi:10.1016/j.pbi.2014.07.009 (2014).
56 Sato, H. et al. Arabidopsis thaliana NGATHA1 transcription factor induces ABA biosynthesis by activating NCED3 gene during dehydration stress. Proc Natl Acad Sci USA 115, E11178-E11187, doi:10.1073/pnas.1811491115 (2018).
57 Hauser, F. et al. A seed resource for screening functionally redundant genes and isolation of new mutants impaired in CO2 and ABA responses. J Exp Bot 70, 641-651, doi:10.1093/jxb/ery363 (2019).
58 Hsu, P. K. et al. Abscisic acid-independent stomatal CO₂ signal transduction pathway and convergence of CO₂ and ABA signaling downstream of OST1 kinase. Proc Natl Acad Sci USA 115, E9971-E9980, doi:10.1073/pnas.1809204115 (2018).
59 Ceciliato, P. et al. Intact leaf gas exchange provides a robust method for measuring the kinetics of stomatal conductance responses to abscisic acid and other small molecules in Arabidopsis and grasses. Plant Methods 15, doi:10.1186/s13007-019-0423-y (2019).
60 Nour-Eldin, H. H., Hansen, B. G., Norholm, M. H. H., Jensen, J. K. & Halkier, B. A. Advancing uracil-excision based cloning towards an ideal technique for cloning PCR fragments. Nucleic Acids Res 34, doi:ARTN e122 10.1093/nar/gkl635 (2006).
61 Wang, C., Zhang, J. & Schroeder, J. I. Two-electrode Voltage-clamp Recordings in Xenopus laevis Oocytes: Reconstitution of Abscisic Acid Activation of SLAC1 Anion Channel via PYL9 ABA Receptor. Bio-protocol 7, doi:10.21769/BioProtoc.2114 (2017).
62 Wang, C. et al. Reconstitution of CO2 Regulation of SLAC1 Anion Channel and Function of CO2-Permeable PIP2; 1 Aquaporin as CARBONIC ANHYDRASE4 Interactor. Plant Cell 28, 568-582, doi:10.1105/tpc.15.00637 (2016).
63 Wu, F. H. et al. Tape-Arabidopsis Sandwich—a simpler Arabidopsis protoplast isolation method. Plant Methods 5, 16, doi:10.1186/1746-4811-5-16 (2009).
64 Gao, X., Chen, J., Dai, X., Zhang, D. & Zhao, Y. An Effective Strategy for Reliably Isolating Heritable and Cas9-Free Arabidopsis Mutants Generated by CRISPR/Cas9-Mediated Genome Editing. Plant Physiol 171, 1794-1800, doi:10.1104/pp. 16.00663 (2016).
65 Gao, Y. et al. Auxin binding protein 1 (ABP1) is not required for either auxin signaling or Arabidopsis development. Proc Natl Acad Sci USA 112, 2275-2280, doi:10.1073/pnas.1500365112 (2015).
66 Gao, Y. & Zhao, Y. Self-processing of ribozyme-flanked RNAs into guide RNAs in vitro and in vivo for CRISPR-mediated genome editing. Journal of integrative plant biology 56, 343-349, doi:10.1111/jipb.12152 (2014).

A number of embodiments of the invention have been described. Nevertheless, it can be understood that various modifications may be made without departing from the spirit and scope of the invention. Accordingly, other embodiments are within the scope of the following claims.

1: A method for:
enhancing or creating drought tolerance of crop plants and trees,
enhancing or creating salinity of tolerance of a plant, wherein optionally the plant is a crop plant,
early monitoring of drought, salinity and cold stress by a plant or a tree, wherein optionally the plant is a crop plant,
enhancing or creating stress resistance in a plant or a tree, wherein optionally the plant is a crop plant,
the method comprising increasing the expression and/or activity of a Raf-like mitogen-activated protein (MAP) kinase kinase (MAPKK) kinase δ B3 family enzyme or an M3K δ B3 family enzyme in the plant or the tree, or a plant cell or a tree cell by:
inserting in the plant or tree, or the plant cell or the tree cell, a heterologous M3K δ B3 family enzyme-expressing nucleic acid,
wherein the nucleic acid is operatively linked to a transcriptional regulatory element that is capable of expressing the M3K δ B3 family enzyme in the plant, tree, plant cell or tree cell,
thereby increasing the amount of M3K δ B3 family enzyme expression or M3K δ B3 family enzyme activity in the plant or tree or plant cell or tree cell.

2: The method of claim 1, wherein the transcriptional regulatory element comprises a promoter.

3: The method of claim 1, wherein the method comprises increasing the total or average amount of M3K δ B3 family enzyme expression or M3K δ B3 family enzyme activity in the plant or tree or plant cell or tree cell by between about 5% and 500%.

4: The method of claim 1, wherein the plant or tree is, or the plant or tree cell is derived from: (i) a dicotyledonous or monocotyledonous plant; (ii) wheat, oat, rye, barley, rice, sorghum, maize (corn), tobacco, a legume, a lupins, potato, sugar beet, pea, bean, soybean (soy), a cruciferous plant, a cauliflower, rape (or rapa or canola), cane (sugarcane), flax, cotton, palm, sugar beet, peanut, a tree, a poplar, a lupin, a silk cotton tree, desert willow, creosote bush, winterfat, balsa, ramie, kenaf, hemp, roselle, jute, or sisal abaca; or, (c) a species from the genera Arabidopsis, Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Man[iota]hot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna or Zea.

5: The method of claim 1, wherein the M3K δ B3 family enzyme or M3K δ B3 family enzyme-expressing nucleic acid is or comprises:
(a) a rice or a plant of the genus Oryza, or a Oryza sativa plant M3K δ B3 enzyme;
(b) a soybean or a plant of the genus Glycine, or a G. max plant M3K δ B3 enzyme; or
(c) a maize or a plant of the genus Zea or a Zea mays plant.

6: The method of claim 1, wherein the heterologous M3K δ B3 family enzyme-expressing nucleic acid is contained in an expression vector.

7: A transgenic guard cell, plant, plant cell, plant tissue, plant seed or fruit, or a plant part or plant organ, that expresses a heterologous M3K δ B3 family enzyme, comprising:
a heterologous M3K δ B3 family enzyme-expressing nucleic acid operatively linked to transcriptional regulatory element.

8: The method of claim 2, wherein the transcriptional regulatory element promoter comprises an inducible promoter, a constitutive promoter, a guard cell specific promoter, a drought-inducible promoter, a stress-inducible promoter or a guard cell active promoter, and optionally the promoter comprises: a pRAB18 drought and ABA-induced promoter; a pGC1 guard cell promoter; a constitutive CAMV 35 promoter; a constitutive pUbi10 promoter.

9: The method of claim 3, wherein the method comprises increasing the total or average amount of M3K δ B3 family enzyme expression or M3K δ B3 family enzyme activity in the plant or tree or plant cell or tree cell by between about 10% and 200%.

10: The method of claim 6, wherein the heterologous M3K δ B3 family enzyme-expressing nucleic acid is contained in an episome.

11: The method of claim 1, wherein the heterologous M3K δ B3 family enzyme-expressing nucleic acid is stably integrated into the plant, tree, plant cell or tree cell genome.

12: The transgenic guard cell, plant, plant cell, plant tissue, plant seed or fruit, or a plant part or plant organ of claim 7, wherein the transcriptional regulatory element comprises a promoter.

13: The transgenic guard cell, plant, plant cell, plant tissue, plant seed or fruit, or a plant part or plant organ of claim 12, wherein the promoter comprises an inducible promoter, a constitutive promoter, a guard cell specific promoter, a drought-inducible promoter, a stress-inducible promoter or a guard cell active promoter.

14: The transgenic guard cell, plant, plant cell, plant tissue, plant seed or fruit, or a plant part or plant organ of claim 13, wherein the promoter comprises: a pRAB18 drought and ABA-induced promoter; a pGC1 guard cell promoter; a constitutive CAMV 35 promoter; a constitutive pUbi10 promoter.

15: The method of claim 1, wherein the M3K δ B3 family enzyme or M3K δ B3 family enzyme-expressing nucleic acid comprises an enzyme-encoding nucleic acid having a sequence as set forth in GenBank accession no. AY167575.1; XP_015625387.1; EEC72758.1; EEE56573.1; BAH01506.1 or XP_015636565.1.

16: The method of claim 1, wherein the M3K δ B3 family enzyme or M3K δ B3 family enzyme-expressing nucleic acid comprises an enzyme-encoding nucleic acid having a sequence as set forth in GenBank accession no. FJ528664.1; XP_003545374; KRH34026.1; ACQ57002.1; XP_006578285.1 or XP_006596381.1.

17: The method of claim 1, wherein the M3K δ B3 family enzyme or M3K δ B3 family enzyme-expressing nucleic acid comprises an enzyme-encoding nucleic acid having a sequence as set forth in GenBank accession no. CM007647.1; XP_008679833.1; AQK59735.1; AQK59729.1; KQJ94060.1 or XP_008668902.1.
